(19) United States
(12) Patent Application Publication
(10) Pub. No.: US 2025/0177290 A1
(43) Pub. Date: Jun. 5, 2025

(54) DRUG-ELUTING SURGICAL ARTICLES AND METHODS OF USE THEREOF

(71) Applicant: BIOSAPIEN INC., San Diego, CA (US)

(72) Inventor: Khatija Pinky Ali, San Diego, CA (US)

(21) Appl. No.: 18/944,843

(22) Filed: Nov. 12, 2024

Related U.S. Application Data
(63) Continuation of application No. PCT/US2023/022089, filed on May 12, 2023.
(60) Provisional application No. 63/342,531, filed on May 16, 2022.
(60) Provisional application No. 63/342,012, filed on May 13, 2022.

(51) Int. Cl. A61K 9/00; A61K 9/48; A61K 31/513; A61P 35/00

(52) U.S. Cl. CPC A61K 9/0024; A61K 9/4808; A61K 9/4816; A61K 31/513; A61P 35/00

(57) ABSTRACT

Disclosed herein is a biomaterial and a method of use thereof for treating a condition. A biomaterial of the disclosure can be, for example, a surgical article. Implantation of a biomaterial disclosed herein into a subject can treat, for example, cancer.

### CROSS REFERENCE

This application claims priority to and the benefit of U.S. Provisional Patent Application Nos. 63/342,012, filed May 13, 2022, and 63/342,531, filed May 16, 2022, each of which is incorporated herein by reference in its entirety.

### STATEMENT AS TO FEDERALLY SPONSORED RESEARCH

This invention was made with government support under 7203584328 awarded by the National Science Foundation. The government has certain rights in the invention.

### BACKGROUND

Implantation of biomaterials can allow for the targeted delivery of a therapeutically-effective amount of a therapeutic agent. Targeted delivery of therapeutic agents can increase agent efficacy by increasing the exposure of target tissues to the agents and by mitigating systemic toxicity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the disclosure provides a biomaterial comprising a first plurality of geometric elements and a therapeutically-effective amount of a therapeutic agent, wherein a first geometric element of the first plurality of geometric elements is formed by a first porous border, wherein the first porous border comprises a polymer and the therapeutic agent, wherein a second geometric element of the first plurality of geometric elements is formed by a non-porous border and a first solid region comprising the polymer, wherein the therapeutic agent cannot diffuse into the second geometric element, wherein the first solid region is adjacent to and within the non-porous border, and wherein a portion of the first porous border is adjacent to a portion of the non-porous border.

In some embodiments, the disclosure provides a method of treating a condition, the method comprising: implanting a biomaterial into a subject, wherein the biomaterial comprises a first plurality of geometric elements and a therapeutically-effective amount of a therapeutic agent, wherein a first geometric element of the plurality of geometric elements is formed by a first porous border, wherein the first porous border comprises a polymer and the therapeutic agent, wherein a second geometric element of the first plurality of geometric elements is formed by a non-porous border and a first solid region comprising the polymer, wherein the therapeutic agent cannot diffuse into the second geometric element from the first porous border, wherein the first solid region is adjacent to and within the non-porous border, and wherein a portion of the first porous border is adjacent to a portion of the non-porous border.

In some embodiments, the disclosure provides a method of treating a condition in a subject in need thereof, the method comprising administering to the subject a biomaterial comprising a plurality of geometric elements and a therapeutic agent, wherein: (a) a first geometric element of the plurality of geometric elements comprises a first polymer and the therapeutic agent, wherein at least a portion of the first geometric element is porous; (b) a second geometric element of the plurality of geometric elements comprises a second polymer, wherein at least a portion of the second geometric element is substantially non-porous; (c) the at least the portion of the first geometric element is adjacent to the at least the portion of the second geometric element; and (d) the first polymer or the second polymer has a number average molar mass of greater than 6,000 Dalton (Da).

In some embodiments, the disclosure provides a method of treating a condition in a subject in need thereof, the method comprising administering to the subject a biomaterial comprising a plurality of geometric elements and a therapeutic agent, wherein: (a) a first geometric element of the plurality of geometric elements comprises a first polymer and the therapeutic agent, wherein an amount of the therapeutic agent in the first geometric element is greater than 30% by weight of the first polymer, and wherein at least a portion of the first geometric element is porous; (b) a second geometric element of the plurality of geometric elements is formed by a second polymer, wherein at least a portion of the second geometric element is substantially non-porous; and (c) the at least the portion of the first geometric element is adjacent to the at least the portion of the second geometric element.

### BRIEF DESCRIPTION OF THE DRAWINGS

FIG. 1A is a line drawing showing the architecture of an example implementation of a single layer of a biomaterial of the disclosure.

FIG. 1B shows a schematic representation of the intra-article diffusion of a therapeutic agent disclosed herein.

FIG. 2A and FIG. 2B show line drawings of a layer of a biomaterial of the disclosure.

FIG. 3 shows a three-layered biomaterial comprising three layers of the single layer biomaterial described in FIG. 1A.

FIG. 4A shows a photograph of an approximately 1×1 cm 3-D printed, PCL-based surgical mesh of the disclosure.

FIG. 4B shows a side view showing the thickness of the 1×1 cm 3-D-printed surgical mesh of FIG. 4A.

FIG. 5 shows a release profile of 5-fluorouracil (5-FU) from a biomaterial of the disclosure. Error bars represent standard deviation from three replicates.

FIG. 6 illustrates the percentage degradation of PF50, PF37, P50 and P37 formulations.

FIG. 7 shows a release profile of 5-FU from a biomaterial.

FIG. 8 shows embodiments of the biomaterial, wherein the darker shading can refer to loading of one or more therapeutic agents (e.g., 5-fluorouracil, gemcitabine).

FIG. 9 illustrates that a biomaterial as disclosed herein can comprise a plurality of portions (e.g., N, S, W, E, etc.) capable of comprising drugs (e.g., 5-fluorouracil, gemcitabine).

FIG. 10 provides a process flow diagram for making a biomaterial.

FIG. 11 outlines a long term stability assessment for a biomaterial.

FIG. 12 outlines an in use stability assessment for a biomaterial.

FIG. 13 is a computer-aided Design (CAD) Image of Biomaterial-5-FU.

FIG. 14 is a photograph of Biomaterial-5-FU.

FIG. 15 summarizes a process of making the biomaterial.

FIG. 16 provides a packaging process for a biomaterial.

FIG. 17 provides a making and quality control flow chart for a biomaterial.

### DETAILED DESCRIPTION

Targeted delivery systems for therapeutic agents can increase the efficacy of a therapeutic agent by, for example, increasing exposure of target tissues to the therapeutic agent, and by mitigating systemic toxicity. Targeted delivery can be desirable in the context of therapies that are highly toxic when administered systemically, and/or when administered over a prolonged period of time, such as chemotherapeutic agents and opioids.

Advances in three-dimensional (3D) printing technologies can create new opportunities for producing customized delivery systems that can be adapted for a wide range of uses in the surgical theater. Three-dimensional printing can be advantageous due to the relatively low cost, simplicity, and versatility of a 3D printing system, as well as the high speed with which custom devices can be produced.

Disclosed herein is a biomaterial that can serve as a surgical article and/or drug delivery system that can release a therapeutic agent directly to a target site over prolonged periods of time. The biomaterial can comprise one or more therapeutic agents that are loaded into a geometric element or a plurality of geometric elements of the article. Biomaterials, such as surgical articles, can be adapted for in situ sustained release of the one or more therapeutic agents. In some embodiments, the surgical articles are printed using an extrusion 3D printing method. Biomaterials can be custom printed in a shape and size suitable to cover a target site in situ. Biomaterials for implantation in situ can be in the general form of a surgical tape or mesh that can also be folded and/or layered. Suitable target sites include any site in the body of a subject requiring treatment with the one or more therapeutic agents. Non-limiting examples of target sites include tissues such as a blood vessel, lymph nodes, cartilage, bone, liver, lungs, heart, pancreas, spleen, gastrointestinal tract, brain, pelvic, breast, and pulmonary tissue. In some embodiments, a biomaterial can be applied to deep tissue and connective tissues such as muscle and smooth muscles.

Biomaterials disclosed herein can comprise a polymer material, within at least a portion of which one or more therapeutic agents can be dispersed. In some embodiments, the polymer material can also contain one or more additives. The polymer material can comprise or consist of one or more bioresorbable and/or biodegradable polymers, or a mixture of polymers including at least one bioresorbable and/or biodegradable polymer.

In some implementations, the 3D printed surgical articles are sterile, sterilizable, and/or sterilized before implantation into a subject.

A biomaterial disclosed herein can be biocompatible. A biocompatible biomaterial can be administered or implanted into the body of a subject without undesirable effects such as, for example, an immune and/or inflammatory reaction.

A biomaterial disclosed herein can be biodegradable. A biodegradable biomaterial can degrade (partially or completely) under physiological conditions into non-toxic products that can be metabolized or excreted from the body. In some instances, biodegradable materials are degraded by enzymatic activity, for example by enzymatic hydrolysis.

In some embodiments, a biomaterial of the disclosure is bioresorbable or bioabsorbable. Bioresorbable or bioabsorbable materials can be broken down and absorbed by cells and/or tissues.

In some embodiments, a biomaterial described herein is configured to resorb and/or degrade after placement in situ over a period of time ranging from about 1 day to about 1 week, about 1 week to about 1 month, 1 month to about 3 months, about 3 months to about 6 months, about 6 months to about 12 months, about 12 months to about 24 months, or from about 2 years to about 5 years. The resorption and/or degradation time can be modulated by controlling the composition of the polymer material, including the types of polymers and porosity of the material, as well as by the two- and three-dimensional arrangement of geometric elements and comprising the article.

A surgical article as described here can comprise one geometric element or a plurality of geometric elements. In some embodiments, the geometric elements of a plurality of geometric elements are in fluid communication with each other. In some embodiments, a plurality of geometric elements is printed on an x-y plan, for example as a ribbon, grid, or other shape to form a biomaterial of a desired shape and/or size. In some embodiments, a plurality of geometric elements can be printed vertically, on top of one another. In some embodiments, a thickness of a single layer article can be in the range of about 0.1 cm to about 1 cm, about 0.25 to about 1 cm, about 0.5 to about 1 cm, about 0.75 to about 1 cm, about 0.1 cm to about 2 cm, about 0.25 to about 2 cm, about 0.5 to about 2 cm, about 0.75 to about 2 cm, about 0.1 cm to about 3 cm, about 0.25 to about 3 cm, about 0.5 to about 3 cm, about 0.75 to about 3 cm, about 0.1 cm to about 4 cm, about 0.25 to about 4 cm, about 0.5 to about 4 cm, about 0.75 to about 4 cm, about 0.1 cm to about 5 cm, about 0.25 to about 5 cm, about 0.5 to about 5 cm, or about 0.75 to about 5 cm.

In some embodiments, a biomaterial of the disclosure comprises multiple layers, wherein each layer comprises a plurality of geometric elements. For example, a biomaterial of the disclosure can comprise 1, 2, 3, 4, 5, 6, 7, 8, 9, 10, or more layers. In some embodiments, each layer of a biomaterial is oriented such that the layer is offset from the unit above and/or below, for example at a defined angle such as, for example, about 15 degrees, about 30 degrees, about 45 degrees, about 60 degrees, about 90 degrees, about 105 degrees, about 120 degrees, about 135 degrees, about 150 degrees, about 165 degrees, or about 180 degrees.

The overall dimensions of a biomaterial of the disclosure can be adapted to accommodate a wide range of in situ target sites. In accordance with any of these implementations, one or more geometric elements of a plurality of geometric elements can be printed with or without one or more therapeutic agents. A biomaterial of the disclosure can have any shape. For example, the overall shape of a surgical article in accordance with the present description can be circular, oval, rectangular, triangular, octangular, pentangular, hexangular, heptangular, or square, and the size can be adapted to cover an area in the range of, for example, from about 4 cm² to about 200 cm². In some embodiments, the article is of a size suitable to cover an area of from about 20 cm² to about 50 cm², from about 50 cm² to about 100 cm², or from about 100 cm² to about 200 cm². In some embodiments, the article can range in size from about 2 cm×about 2 cm up to about 12 cm×about 10 cm. For example, a biomaterial of the disclosure can be a 2 cm×2 cm, 4 cm×4 cm, 6 cm×6 cm, or 8 cm×8 cm square article, or 4 cm×6 cm, 8 cm×6 cm, 10 cm×8 cm, or 12 cm×10 cm rectangular article.

In some embodiments, a biomaterial of the disclosure (e.g. a surgical article) can further comprise a loop or similar feature configured to facilitate the placement of the biomaterial in situ, for example by suturing.

A biomaterial disclosed herein can have a flexible structure. The biomaterial can be a dosage form that is locally administered using a minimally invasive/endoscopic procedure. The biomaterial can allow 5-FU to directly target tumor cells at hard to reach tumor sites. This site-specific delivery technology has promise for reducing side effects caused by systemic therapy. The biomaterial can downsize localized tumors, alleviate local symptoms, and potentially prevent or reduce the likelihood of life-altering surgeries such as colostomy.

In some embodiments a biomaterial disclosed herein is radiopaque. In some embodiments a biomaterial disclosed herein contains no or substantially no additives or fillers other than a drug and excipient (e.g., PCL) disclosed herein. In some embodiments, a biomaterial releases an active pharmaceutical ingredient (API) from pores within the biodegradable polymer in a slow-release (e.g., biphasic-release) manner over a period of 4-weeks. The release can be enhanced by the hydrophobicity of the product polymer.

### Structure of Geometric Elements.

A biomaterial of the disclosure can comprise a plurality of geometric elements. Geometric elements of a plurality of geometric elements can be adjacent and/or in fluidic communication with one another. In some embodiments, the geometric elements form a layer. Geometric elements can be formed by a border comprising a polymer. In some embodiments, a border of a geometric element can further comprise one or more therapeutic agents disclosed herein. A border forming a geometric element can be porous, non-porous, or minimally porous. A porous border can allow for the diffusion of a therapeutic agent through the border and into or out of the geometric element formed by the border. In some embodiments, a border is minimally porous such that a therapeutic agent cannot diffuse through the border and into the geometric element formed by the border.

A geometric element formed by a border can comprise, for example, an empty space or a solid region within the border. A solid region of a geometric element can be porous, non-porous, or minimally porous. A porous solid region can allow for the diffusion of a therapeutic agent through the solid region and into or out of a geometric element. In some embodiments, a solid region is minimally porous such that a therapeutic agent cannot diffuse through the solid region and into the geometric element formed by the border.

A border and/or solid region of a geometric element can have a degree of porosity. In some embodiments, the degree of porosity of a border or solid region is about 10% to about 99%. In some embodiments, the degree of porosity of a border or solid region is about 10% to about 20%, about 10% to about 30%, about 10% to about 40%, about 10% to about 50%, about 10% to about 60%, about 10% to about 70%, about 10% to about 80%, about 10% to about 90) %, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%. In some embodiments, the degree of porosity of a border or solid region is about 10%, about 20%, about 30%, about 40%, about 50%, about 60%, about 70%, about 80%, about 90%, or about 99%. In some embodiments, the degree of porosity of a border or solid region is at least about 10%, at least about 20%, at least about 30%, at least about 40%, at least about 50%, at least about 60%, at least about 70%, at least about 80%, or at least about 90%. In some embodiments, the degree of porosity of a border or solid region is at most about 20%, at most about 30%, at most about 40%, at most about 50%, at most about 60%, at most about 70%, at most about 80%, at most about 90%, or at most about 99%.

The porosity of borders and/or solid regions can vary throughout a biomaterial of the disclosure. In some embodiments, a porous border surrounds a solid region that is minimally porous or non-porous. In some embodiments, a minimally porous or non-porous border surrounds a solid region that is porous. In some embodiments, a non-porous or minimally porous border surrounds a solid region that is non-porous or minimally porous. In some embodiments, a porous border surrounds a porous solid region.

Adjacent geometric elements can share an edge, and thus form a portion of the border forming the geometric elements. In some embodiments, adjacent elements each comprise distinct edges/borders that are adjacent to and/or in contact with one another.

Borders can form geometric elements of any shape. For example, a border can form a geometric element that is circular, elliptical, triangular, rectangular, pentangular, hexangular, heptangular, octangular, or irregularly shaped. In some embodiments, a border forms a geometric element that is a nonagon or decagon. Geometric elements of a plurality of geometric elements can be the same or different shapes. In some embodiments, a biomaterial or layer thereof can comprise a plurality of geometric elements defining two or more hexagons, triangles, and diamonds, or portions thereof. In some implementations, the length of the edges of a particular element may range from 1.0 mm to 10 mm, or from 1.0 mm to 5 mm, or from 1.0 mm to 3 mm. It is understood that, depending on the geometric shape defined by the edges of the element, the edges may be the same or different lengths. In some embodiments, each edge of a geometric element is uniform in length and/or width.

In some embodiment, a biomaterial disclosed herein is comprised of a plurality of layers, each layer formed from a plurality of open and filled geometric elements forming a defined pattern. The plurality of geometric elements can comprise elements having three, four, five, or six edges. In some embodiments, the edges of the elements can define one or more geometric shapes selected from triangles, diamonds, hexagons, and portions of any of the foregoing. In some embodiments, the triangles are equilateral triangles and the edges of each geometric element are of uniform length. In some embodiments, the edges have a length of from about 1 to about 3 mm, from about 1 to about 2 mm, for example about 1.0 mm, about 1.5 mm, about 2.0 mm, about 2.5 mm, or about 3.0 mm.

One or more therapeutic agents can be loaded into geometric elements. For example, one or more geometric agents can be dispersed within a solid region or a border of a one or more geometric elements of a biomaterial of the disclosure.

### Arrangement of Geometric Elements.

In some embodiments, a plurality of geometric elements forming a biomaterial or layer thereof here is arranged in a defined pattern of elements comprising solid regions (i.e., filled elements) and empty spaces (i.e., open elements), the pattern adapted to modulate release of the therapeutic agent(s) from the drug reservoir element(s) of the unit article in situ. For example, the size and shape of the elements forming the biomaterial or layer thereof can be increased or decreased to modulate the surface area of the filled elements and the pore size of the open elements, in order to modulate the release of the therapeutic agent or agents from the article. The volume and number of therapeutic-agent loaded element(s) can determine the amount of the therapeutic agent(s) in the unit article and the amount released in situ at the target site. The volume of the therapeutic-agent loaded element(s) can be increased, for example, by printing multiple single layers of biomaterial on top of each other until a desired thickness is reached, thereby allowing a higher loading volume for the therapeutic agent(s). The defined pattern of open and filled geometric elements can further be used to modulate the total surface area of the unit article, as well as the surface area of the unit article from which a therapeutic agent(s) is released.

### Release of Therapeutic Agents.

Release of one or more therapeutic agents into a target site in situ from a biomaterial of the disclosure can occur from the exposed surface(s) of the biomaterial. Additionally, or alternatively, one or more therapeutic agents can diffuse within a biomaterial disclosed herein.

For example, in some embodiments, a defined pattern of open and filled geometric elements with porous and/or non-porous components (e.g. borders or solid regions) can be used to direct or focus any intra-biomaterial diffusion of a therapeutic agent to a defined region of the biomaterial. Focusing diffusion of a therapeutic agent can have the effect of a more concentrated release of a therapeutic agent from a region of a biomaterial. Thus, depending on the type of therapy, duration of treatment, and the location of an in situ target site, the amount and rate of release of therapeutic agent(s) from a biomaterial described herein can be controlled through a combination of the defined pattern of open and filled geometric elements forming the biomaterial, the inclusion of one or more additives, such as a poragen, and the amount of the agent(s) loaded into the geometric elements of the biomaterial.

In some embodiments, release of a therapeutic agent from a biomaterial disclosed herein can be modulated by increasing the thickness of the biomaterial (e.g., by using multiple layers) as well as by sequestering the geometric element(s) loaded with therapeutic agent within internal layers of a folded biomaterial or a stack of biomaterials or layers thereof. Folding and stacking of the biomaterials of layers thereof in this manner can reduce the surface area from which a therapeutic agent can be released. In some embodiments, a surgical article can be formed from alternating layers of unit articles in a stacked configuration where the alternating layers are staggered or slanted, for example at about 180 degrees, about 90 degrees or about 45 degrees. For example, in the staggered configuration, two or more layers of a biomaterial are layered horizontally on top of one another at a 180 degree. 90 degree, or 45 degree angle. In some embodiments, the thickness of a folded or stacked article can range from about 0.5 cm to about 3 cm. In some embodiments, the thickness of the folded or stacked article is about 0.5 cm, about 1.0 cm, about 1.5 cm, or about 2.0 cm.

In some embodiments, one or more therapeutic agents can be coated onto the surface of a biomaterial in addition to, or instead of, being dispersed within the polymer material of the biomaterial.

### Biomaterial Structure.

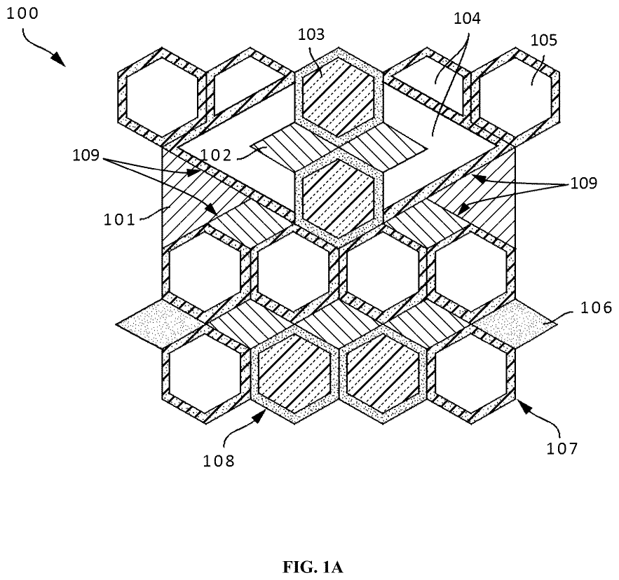

A schematic of a non-limiting example of a biomaterial of the disclosure is shown in FIG. 1A. As shown in FIG. 1A, a biomaterial of the disclosure (100) can comprise twenty-seven open (i.e. filled with empty space) or filled elements defining five different geometric shapes and portions thereof. Two filled triangular elements (101) and seven filled diamond shaped elements (102) can be positioned to modulate diffusion of a therapeutic agent infused within portions of the biomaterial, including within adjacent filled hexagonal elements (103). In some embodiments, the filled elements each contain the same polymer or mixtures of polymers. The filled elements can contain polymers or mixtures of polymers with the same or different porosities. For example, some of the filled elements can function as a barrier to intra-material diffusion of the therapeutic agent, while other filled elements can contain pores that serve to allow intra-material diffusion of the therapeutic agent into the area defined by the porous filled elements. The degree of porosity of polymers making up the biomaterial can be modulated by the manufacturing process.

In FIG. 1A, the filled triangular elements (101) are formed by a non-porous border (109) around an essentially non-porous solid region. The filled triangular elements (109) can serve to focus the intra-material diffusion of the therapeutic agent into adjacent regions defined by porous or open elements, for example the filled hexagonal elements (103), the open hexagonal elements, (105), and/or the open triangular elements and portions thereof (104). The focusing of the intra-material diffusion can, for example, target release of a therapeutic agent toward a defined region of the biomaterial for release in a more concentrated fashion at a particular region within a target site in situ.

As shown in FIG. 1A, a biomaterial disclosed herein can comprise four filled hexagonal elements (103) formed by a porous border (108) and a solid region, each comprising polymer and the therapeutic agent dispersed within the polymer. The biomaterial can further comprise two filled diamond-shaped elements (106) formed by a solid region and a porous border, each comprising polymer and the therapeutic agent dispersed within the polymer. In some embodiments, the porous border is formed by the edges of the solid region. Throughout the biomaterial, the border of a geometric element with a solid region can have a composition that is the same or different than the composition of the solid region. In some embodiments, a biomaterial contains diamond-shaped filled elements (102) formed by a porous border and a porous solid region that is not infused with therapeutic agent, but which allows for intra-material diffusion of the therapeutic agent into these diamond-shaped filled elements (102). Diffusion of the therapeutic agent into the non-therapeutic agent infused diamond-shaped elements (102) increases the surface area from which the therapeutic agent diffuses into the in situ target site.

A biomaterial disclosed herein can further comprise a plurality of filled and open elements that serve to increase the overall surface area of the biomaterial, impart structural integrity to the biomaterial, impart flexibility to the bio biomaterial, and/or serve as optional fixation points on the biomaterial. For example, as shown in FIG. 1A, open elements (104, 105) of a biomaterial disclosed herein can serve as fixation points to suture the biomaterial in place at a target site. The borders (107) forming the open elements (104, 105) can be porous or nonporous borders. In some embodiments the borders forming the open elements (104, 105) of a biomaterial disclosed herein comprise a polymer and a therapeutic agent (e.g. 5-fluorouracil) infused within the polymer.

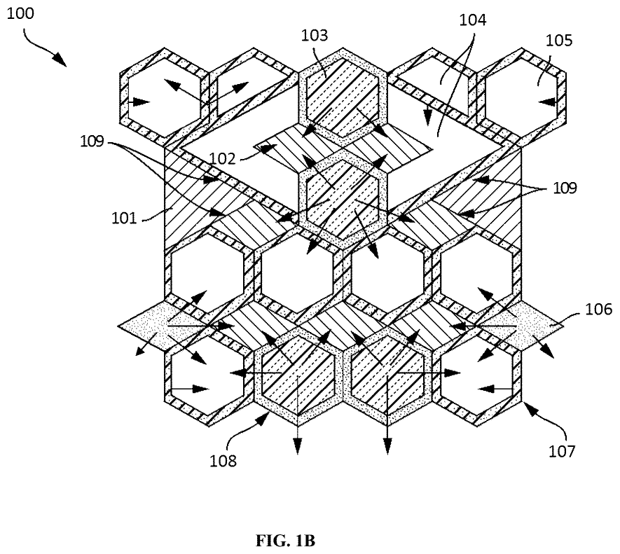

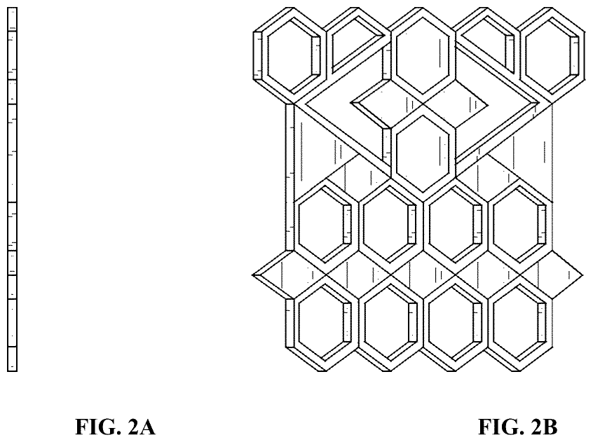

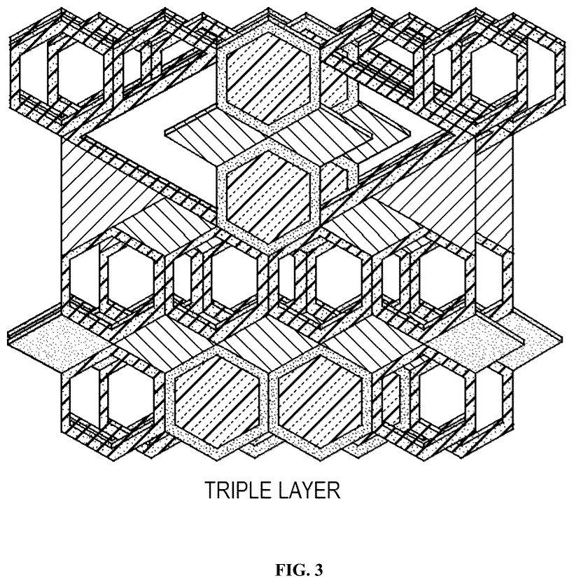

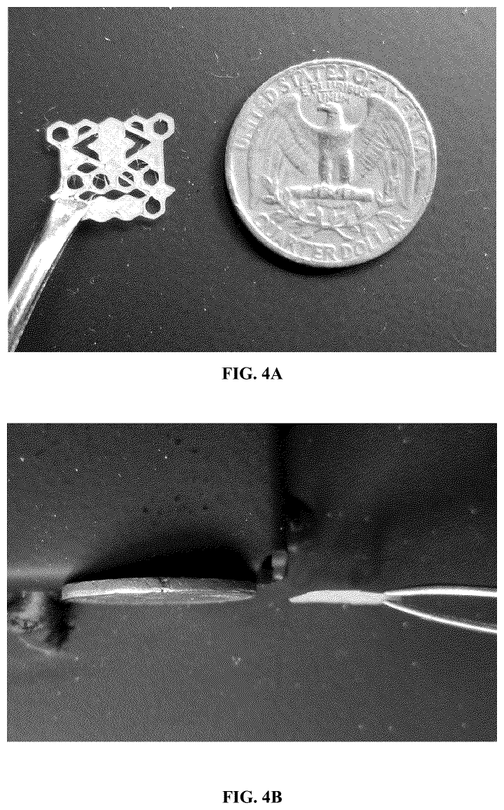

A biomaterial disclosed herein can be one or multiple layers. For example, a biomaterial of the disclosure can be 1, 2, 3, 4, 5, 6, 7, 8, 9, 10, or more layers. A non-limiting example of a single layer biomaterial is shown in FIG. 2A and FIG. 2B. FIG. 2A shows a head-on view, while FIG. 2B and shows a rotated view of a single layer article. A non-limiting example of a multiple layer biomaterial is shown in FIG. 3. FIG. 3 depicts a layered biomaterial containing three layers of the biomaterial described in FIG. 1, with each layer offset by about 30 degrees.

### Intra-Material Diffusion.

In some embodiments, a biomaterial disclosed herein modulates the diffusion of a therapeutic agent within and from the biomaterial. A non-limiting example of an intra-material diffusion pattern of a therapeutic agent through a biomaterial disclosed herein (the biomaterial depicted in FIG. 1A) is shown in FIG. 1B. In FIG. 1A and FIG. 1B, filled hexagonal elements (103) and diamond shaped elements (b) contain the bulk of the therapeutic agent per unit of the biomaterial, although the edges defining the elements also contain therapeutic agent. In some embodiments, the six filled hexagonal elements can contain a total of 50-500 mg, 100-400 mg, or 150-250 mg of the therapeutic agent. Each element can contain the same or a different amount of the therapeutic agent. In some embodiments, the therapeutic agent diffuses into the region defined by filled diamond-shaped elements (102), which have a degree of porosity that is conducive to such diffusion. In some embodiments, a biomaterial disclosed herein further comprises diamond-filled elements infused with a therapeutic agent (106) that serve as additional reservoirs of the therapeutic agent.

### Polymers.

Biomaterials described herein can be formed from a 3D printed polymer material. In some embodiments, the polymer material can comprise bioresorbable and/or biodegradable polymers, or a mixture of polymers including one or more bioresorbable and/or biodegradable polymers. Suitable polymers include, but are not limited to, polycaprolactone (PCL), poly(lactic acid) (PLA), poly(lactic-co-glycolic acid), which may also be referred to interchangeably as poly(lactide-co-glycolide) (PLGA), poly(ethylene glycol) diacrylate (PEGDA) and poly(ester amide) copolymers (PEA).

In some embodiments, the polymer material comprises or consists of PCL. In some embodiments, the polymer material is a blend of PCL with one or more additional polymers. In some embodiments, the one or more additional polymers blended with the PCL is selected from PLA, PLGA, and PEGDA. In some embodiments, the one or more additional polymers blended with the PCL is selected from polyvinyl chloride and polyethylene oxide (PEO), or from PEA, polyesters, poly(alpha-hydroxy acids), polylactones, polyorthoesters, polycarbonates, polyanhydrides, polyphosphazenes, or a gelatin based polymer such as poly(ethylene glycol) (PEG)-gelatin methacrylate.

In some embodiments, the polymer material is a blend of PCL and PLGA, for example a blend of 1:1 to 10:1 PCL:PLGA. In some embodiments, the polymer material comprises a 1:1-5:1 mixture of PCL/PLGA, a 1:1-2:1 mixture of PCL/PLGA. The ratio of lactide:glycolide of the PLGA may also be varied to modulate release of the therapeutic agent and degradation time of the article in situ. The percentage of PCL in the blend can determine the density of the co-polymer and the ratio of PCL to PLGA can indicate the number of elongated (stretched) polymer fibers present in the co-polymer. An increased number of elongated fibers throughout the polymeric article can allow greater drug release from the article. In some embodiments, the PLGA % weight ratio is from 50:50-90:10 lactide/glycolide. In some embodiments, the PLGA % weight ratio is 85:15 lactide/glycolide, 60:40 lactide/glycolide.

Modulation of the density and/or porosity of a polymer material can modulate the release of a therapeutic agent from a biomaterial. For example, release of a therapeutic agent can be increased by increasing the pore size and/or increasing the porosity of the polymer material. In some embodiments, the polymer material comprises or consists of PCL having micropores in the range of 50-250 microns in size, with an average size of about 80 microns. Blending the PCL with another polymer, such as PLA, PLGA, PEGDA, or PEO, yields larger pores, for example in the range of 200 to 800 microns. In some embodiments, a less porous polymer material can be used to slow release of a therapeutic agent. For example, a polymer material that comprises from about 60% to 100% PCL, or from 60-80% PCL, or from 80-100% PCL can be used.

In some embodiments, a biomaterial of the disclosure can comprise geometric elements that each comprise two or more different polymer materials. For example, the polymer material forming the element edges can differ from the polymer material forming the “fill” of a filled element, and different filled elements can be filled with different polymer materials. The polymer materials can differ, for example, in the type of polymer or mixture of polymers making up the polymer material. Additionally, the polymer materials forming the different portions of geometric elements can have different densities and/or porosities and can also differ in the optional additives they contain. For example, a multi-head 3D printer can allow for different substances to be printed simultaneously. For example, each head can contain different components so one head can contain the polymer and therapeutic agent of choice and a second head can contain only polymer. As an STL file is converted into G-code, programmable code for the multi-head printer is created allowing the 3D printer to read which article segments contain PCL and which article segments contain PCL and drug (as programmed). Similarly, more than one drug can be printed at a time. For example, an analgesic (e.g., NSAID) can be printed simultaneously with a chemotherapy drug (e.g. 5-fluorouracil).

In some embodiments, a biomaterial comprises a polymer (e.g., a first polymer and/or a second polymer disclosed herein), and the polymer has a number average molar mass of at least 0.5 kilodalton (kDa), at least 1 kDa, at least 2 kDa, at least 5 kDa, at least 7 kDa, at least 10 kDa, at least 20 kDa, at least 30 kDa, at least 40 kDa, at least 50 kDa, at least 70 kDa, at least 100 kDa, at least 200 kDa, at least 500 kDa, at least 700 kDa, or at least 1,000 kDa.

In some embodiments, a biomaterial comprises a polymer (e.g., a first polymer and/or a second polymer disclosed herein), and the polymer has a number average molar mass of at most 1 kilodalton (kDa), at most 2 kDa, at most 5 kDa, at most 7 kDa, at most 10 kDa, at most 20 kDa, at most 30 kDa, at most 40) kDa, at most 50 kDa, at most 70 kDa, at most 100 kDa, at most 200 kDa, at most 500 kDa, at most 700 kDa, or at most 1,000 kDa.

In some embodiments, a biomaterial comprises a polymer (e.g., a first polymer and/or a second polymer disclosed herein), and the polymer has a number average molar mass of about 1 kilodalton (kDa), about 2 kDa, about 5 kDa, about 7 kDa, about 10 kDa, about 20 kDa, about 30) kDa, about 40) kDa, about 50) kDa, about 70) kDa, about 100 kDa, about 200) kDa, about 500 kDa, about 700 kDa, or about 1,000 kDa.

In some embodiments, a biomaterial comprises a first polymer and a second polymer, and the first polymer and the second polymer each have a number average molar mass of at least 0.5 kilodalton (kDa), at least 1 kDa, at least 2 kDa, at least 5 kDa, at least 7 kDa, at least 10 kDa, at least 20 kDa, at least 30 kDa, at least 40 kDa, at least 50 kDa, at least 70 kDa, at least 100 kDa, at least 200 kDa, at least 500 kDa, at least 700 kDa, or at least 1,000 kDa.

In some embodiments, a biomaterial comprises a first polymer and a second polymer, and the first polymer and the second polymer each have a number average molar mass of at most 1 kilodalton (kDa), at most 2 kDa, at most 5 kDa, at most 7 kDa, at most 10 kDa, at most 20 kDa, at most 30 kDa, at most 40 kDa, at most 50 kDa, at most 70 kDa, at most 100) kDa, at most 200 kDa, at most 500 kDa, at most 700 kDa, or at most 1,000 kDa.

In some embodiments, a biomaterial comprises a first polymer and a second polymer, and the first polymer and the second polymer each have a number average molar mass of about 1 kilodalton (kDa), about 2 kDa, about 5 kDa, about 7 kDa, about 10 kDa, about 20 kDa, about 30 kDa, about 40 kDa, about 50 kDa, about 70 kDa, about 100 kDa, about 200 kDa, about 500 kDa, about 700 kDa, or about 1,000 kDa.

### Additives.

In some embodiments a biomaterial of the disclosure, or portions thereof, can comprise one or more additives. Non-limiting examples of additives include a radiopaque agent, a colorant, an oil (e.g., silicone) and a porogen.

The density and/or porosity of the polymer material, and therefore the release of the therapeutic agent from the surgical article, can be modulated by including one or more additives, such as a porogen, in the polymer material. In some embodiments, the polymer material, or at least a portion of the polymer material, comprises a porogen. The term “porogen” refers to a material that diffuses, dissolves, and/or degrades leaving pores within the polymer material. In some embodiments, a 3D printed surgical article as described herein can be printed with at least a portion of polymer material comprising a porogen. Depending on the porogen used, the porogen can subsequently, either prior to implantation, or after implantation, diffuse, dissolve, and/or degrade, leaving behind pores in the surgical article. Non-limiting examples of porogens include, water soluble materials such as salts, polysaccharides, water soluble inorganic materials such as bioactive glass, silicate-based nanoparticles, such as lithium sodium magnesium silicate (Laponite™) and water soluble or physiologically labile natural or synthetic polymers, including for example, poly(vinylpyrrolidone), pullulan, poly(glycolide), poly(lactide), poly(lactide-co-glycolide), other polyesters, and starches.

In some embodiments, a porogen of the disclosure is a bioactive glass such as a ceramic within the Na—Ca—Si—P—O system. In embodiments, the bioactive glass comprises SiO₂ and CaO. In embodiments, the bioactive glass further comprises Na₂O and P₂O₅. In some embodiments, the bioactive glass is selected from Bioglass®, bioactive glass 45S5 (45 wt % SiO₂, 24.5 wt % CaO, 24.5 wt % Na₂O and 6.0 wt % P₂O₅), bioactive glass 58S, 60 wt % SiO₂, 36 wt % CaO and 4 wt % P₂O₅, bioactive glass 70S30C, 70 wt % SiO₂, and 30 wt % CaO, bioactive glass S53P₄, 53 wt % SiO₂, 23 wt % Na₂O, 20 wt % CaO and 4 wt % P₂O₅ (anti-bacterial), and laponite (Na⁺_{0.7} (Si₈Mg_{5.5}Li_{0.3})O₂₀(OH)₄]⁻.

In some embodiments, a sacrificial or fugitive ink can be used to introduce pores or channels into a polymer material. Non-limiting examples of materials that can serve as fugitive inks include poloxamers, such as Pluronic™ F127, which consists of hydrophobic poly(propylene oxide) (PPO) and hydrophilic poly(ethylene oxide) (PEO) segments arranged in a PEO-PPO-PEO configuration.

### Therapeutic Agents.

Biomaterials described here can comprise a therapeutically-effective amount of one or more therapeutic agents. In some embodiments, therapeutic agents are loaded into solid regions of geometric elements of the biomaterial. In some embodiments, the therapeutic agent(s) can be contained within the borders (e.g. a porous border) forming the edges of one or more geometric elements of the article.

In some embodiments, the one or more therapeutic agents may be selected from an anti-cancer agent, an antimicrobial agent, an antibiotic, a local anesthetic or analgesic, a statin, and an anti-inflammatory agent.

In some embodiments, the anti-cancer agent is selected from capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, vinorelbine, and combinations thereof.

In some embodiments, the antimicrobial agent is an antibiotic. In some embodiments, the antibiotic can be a broad spectrum antibiotic, such as gentamicin, clindamycin, and erythromycin, or a gram positive and gram negative family antibiotic such as an ampicillin and a cephalosporin. Non-limiting examples of antibiotics suitable for the uses herein include penicillin V potassium, cloxacillin sodium, dicloxacillin sodium, oxacillin sodium, carbenicillin indanyl sodium, oxytetracycline, hydrochloride, tetracycline hydrochloride, clindamycin phosphate, clindamycin, hydrochloride, clindamycin palmitate HCL, lincomycin HCL, novobiocin sodium, nitrofurantoin sodium, and metronidazole hydrochloride.

In some embodiments, a therapeutic agent is a local anesthetic or analgesic. Non-limiting examples of local anesthetics or analgesics include lidocaine, bupivacaine, tetracaine, ropivacaine, benzocaine, and fentanyl, codeine hydrochloride, codeine phosphate, codeine sulphate, dextromoramide tartrate, hydrocodone bitartrate, hydromorphone hydrochloride, pethidine hydrochloride, methadone hydrochloride, morphine sulphate, morphine acetate, morphine lactate, morphine meconate, morphine nitrate, morphine monobasic phosphate, morphine tartrate, morphine valerate, morphine hydrobromide, morphine hydrochloride, and propoxyphene hydrochloride.

In some embodiments, a therapeutic agent is an anti-inflammatory agent. An anti-inflammatory agent can be selected from a non-steroidal anti-inflammatory agent. Non-limiting examples of non-steroidal anti-inflammatory agents include choline salicylate, ibuprofen, ketoprofen, magnesium salicylate, meclofenamate sodium, naproxen sodium, and tolmetin sodium. In some implementations, the one or more anti-inflammatory substances is selected from a non-specific anti-inflammatory such as ibuprofen and aspirin, or a COX-2 specific inhibitor such as rofecoxib and celecoxib.

In some embodiments, a therapeutic agent is an anti-cancer agent. In some embodiments, an anti-cancer agent is selected from an agent used in treating colorectal cancer. In some embodiments, the anti-cancer agent is selected from gemcitabine (Gemzar™), raltitrexed (Tomudex™) oxaliplatin (Eloxatin™), regorafenib, irinotecan (Camptostar™), and 5-fluorouracil (Adrucil™), and combinations thereof. In some embodiments, the anti-cancer agent is selected from capecitabine, fluorouracil, irinotecan and oxaliplatin, and combinations thereof.

In some embodiments, the anti-cancer agent is selected from an agent used in treating pancreatic cancer. In some embodiments, the anti-cancer agent is selected from gemcitabine (Gemzar™), fluorouracil (5-FU), irinotecan (Camptosar™), oxaliplatin (Eloxatin™), paclitaxel (Taxol™ or Abraxane™), capecitabine (Xeloda™) cisplatin, docetaxel (Taxotere™), and irinotecan (Onivyde™), and combinations thereof.

In some embodiments, the anti-cancer agent is selected from an agent used in treating lung cancer. In some embodiments, the anti-cancer agent is selected from cisplatin (Platinol™), carboplatin (Paraplatin™), docetaxel (Taxotere™), gemcitabine (Gemzar™), paclitaxel (Taxol™ and others), vinorelbine (Navelbine™ and others), pemetrexed (Alimta™), and combinations thereof.

In some embodiments, the anti-cancer agent is selected from an agent used in treating bone cancer. In some embodiments, the anti-cancer agent is selected from doxorubicin (Adriamycin®), cisplatin, etoposide (VP-16), ifosfamide (Ifex®), cyclophosphamide (Cytoxan®), methotrexate, and vincristine (Oncovin®), and combinations thereof.

In some embodiments, a therapeutic agent is a cell (e.g. a human cell). For example, the one or more therapeutic agents of a biomaterial can be selected from, pluripotent stem cells, multipotent stem cells, and induced pluripotent stem cells (iPSCs).

In some embodiments, a biomaterial comprises a geometric element (e.g., a first or second geometric element disclosed herein), a polymer (e.g., a first or second polymer disclosed herein), and a therapeutic agent, and an amount of the therapeutic agent in the biomaterial, geometric element, or polymer is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of the biomaterial, geometric element, or polymer.

In some embodiments, a biomaterial comprises a geometric element (e.g., a first or second geometric element disclosed herein), a polymer (e.g., a first or second polymer disclosed herein), and a therapeutic agent, and an amount of the therapeutic agent in the biomaterial, geometric element, or polymer is at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% by weight of the biomaterial, geometric element, or polymer.

In some embodiments, a biomaterial comprises a geometric element (e.g., a first or second geometric element disclosed herein), a polymer (e.g., a first or second polymer disclosed herein), and a therapeutic agent, and an amount of the therapeutic agent in the biomaterial, geometric element, or polymer is about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the biomaterial, geometric element, or polymer.

In some embodiments, a biomaterial comprises a geometric element (e.g., a first or second geometric element disclosed herein), a first polymer, a second polymer, and a therapeutic agent, and an amount of the therapeutic agent in the biomaterial, geometric element, or polymer is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of a combination of the first polymer and the second polymer.

In some embodiments, a biomaterial comprises a geometric element (e.g., a first or second geometric element disclosed herein), a first polymer, a second polymer, and a therapeutic agent, and an amount of the therapeutic agent in the biomaterial, geometric element, or polymer is at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% by weight of a combination of the first polymer and the second polymer.

In some embodiments, a biomaterial comprises a geometric element (e.g., a first or second geometric element disclosed herein), a first polymer, a second polymer, and a therapeutic agent, and an amount of the therapeutic agent in the biomaterial, geometric element, or polymer is about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of a combination of the first polymer and the second polymer.

### Methods of Printing Biomaterials.

Also disclosed herein are methods for printing biomaterials of the disclosure. In some embodiments, the biomaterials are printed using an extrusion-based process. Extrusion-based 3D printing can include any of: fused filament fabrication (FFF), fused deposition modeling (FDM), stereolithography, and gel mediums (with or without granules).

Printing of a biomaterial can comprise heating a polymer material the polymer's melting point and combining the polymer with a therapeutic agent, with or without additional ingredients, such as a porogen, to create a suspension of the therapeutic agent in the polymer material. The resulting combination, which is referred to here as a “slurry” can then loaded into a printing head of an extrusion-based 3D printer. The printing head can be, for example, a syringe. Variations of this process include, but are not limited to, combining the polymer material with the therapeutic agent and then heating the combination to the melting point of the polymer material to form the slurry. Additional ingredients, such as a porogen, can be added at any time during the process. The slurry is extruded onto a substrate along the pre-designed path to form a biomaterial as described herein using a layer by layer process. In some embodiments, the model for the biomaterial is obtained by computer aided design (CAD).

In some embodiments, biomaterials of the disclosure comprise multiple layers. Each layer can have a different geometry (e.g., staggered, slanted). Layered biomaterials can be constructed using a layer-by-layer process to achieve an overall mesh size of, for example, about 4× about 6 cm×about 0.5 cm (h×w×d).

In some embodiments, a biomaterial article as described herein is printed in layers using alternating layers of polymer material with and without the one or more therapeutic agents dispersed in the polymer. For example, (i) a first layer of polymer material containing a therapeutic agent dispersed in the polymer, (ii) a second layer of polymer material without the therapeutic agent, the second layer disposed on the first layer, and (iii) a third layer of polymer material containing the therapeutic agent dispersed in the polymer, the third layer disposed on the second layer. The layers are printed in this sequence, repeating until the article has attained a desired thickness. In some implementations, the article can have a thickness in the range of from 0.5 to 3.0 cm or from 0.5 to 2.0 cm, or from 0.5 to 1.0 cm.

### Bioprinting Parameters

A method disclosed herein can utilize a needle in a bioprinting process. In some embodiments, one or more polymeric materials, fugitive inks, ECM materials, cell suspensions, or a combination thereof is deposited through a needle onto a substrate. A method of the disclosure can print from more than one needle, for example, 1, 2, 3, 4, 5, 6, 7, 8, 9, 10, 11, 12, 13, 14, 15 or more needles. In some embodiments, a needle used in a method disclosed herein has a diameter that is expressed using the Birmingham gauge system. In some embodiments, a needle has a diameter of 7 gauge. 8 gauge, 9 gauge, 10 gauge, 11 gauge, 12 gauge, 13 gauge, 14 gauge, 15 gauge, 16 gauge, 17 gauge, 18 gauge, 19 gauge, 20) gauge, 21 gauge, 22 gauge, 22 s gauge, 23 gauge, 24 gauge, 25 gauge, 26 gauge, 26 s gauge, 27 gauge, 28 gauge, 29 gauge, 30) gauge, 31 gauge, 32 gauge, 33 gauge, or 34 gauge.

In some embodiments, a needle of the disclosure has a diameter of between 0.1 mm to 400 mm. In some embodiments, a needle of the disclosure has a diameter of between 0.1 mm to 0.5 mm, between 0.1 mm to 1 mm, between 0.1 mm to 10 mm, between 0.1 mm to 20 mm, between 0.1 mm to 30 mm, between 0.1 mm to 40 mm, between 0.1 mm to 50 mm, between 0.1 mm to 100 mm, between 0.1 mm to 200 mm, between 0.1 mm to 300 mm, between 0.1 mm to 400 mm, between 0.5 mm to 1 mm, between 0.5 mm to 10 mm, between 0.5 mm to 20 mm, between 0.5 mm to 30 mm, between 0.5 mm to 40 mm, between 0.5 mm to 50 mm, between 0.5 mm to 100 mm, between 0.5 mm to 200 mm, between 0.5 mm to 300 mm, between 0.5 mm to 400 mm, between 1 mm to 10 mm, between 1 mm to 20 mm, between 1 mm to 30 mm, between 1 mm to 40 mm, between 1 mm to 50 mm, between 1 mm to 100 mm, between 1 mm to 200 mm, between 1 mm to 300 mm, between 1 mm to 400 mm, between 10 mm to 20 mm, between 10 mm to 30 mm, between 10 mm to 40 mm, between 10 mm to 50 mm, between 10 mm to 100 mm, between 10 mm to 200 mm, between 10 mm to 300 mm, between 10 mm to 400 mm, between 20 mm to 30 mm, between 20 mm to 40 mm, between 20 mm to 50 mm, between 20 mm to 100 mm, between 20 mm to 200 mm, between 20 mm to 300 mm, between 20 mm to 400 mm, between 30 mm to 40 mm, between 30 mm to 50 mm, between 30 mm to 100 mm, between 30 mm to 200 mm, between 30 mm to 300 mm, between 30 mm to 400 mm, between 40 mm to 50 mm, between 40 mm to 100 mm, between 40 mm to 200 mm, between 40 mm to 300 mm, between 40 mm to 400 mm, between 50 mm to 100 mm, between 50 mm to 200 mm, between 50 mm to 300 mm, between 50 mm to 400 mm, between 100 mm to 200 mm, between 100 mm to 300 mm, between 100 mm to 400 mm, between 200 mm to 300 mm, between 200 mm to 400 mm, or between 300 mm to 400 mm. In some embodiments, a needle of the disclosure has a diameter of 0.1 mm, 0.2 mm, 0.5 mm, 1 mm, 10 mm, 20 mm, 30 mm, 40 mm, 50 mm, 100 mm, 200 mm, 300 mm, or 400 mm. In some embodiments, a needle of the disclosure has a diameter of at least 0.1 mm, at least 0.5 mm, at least 1 mm, at least 10 mm, at least 20 mm, at least 30) mm, at least 40) mm, at least 50 mm, at least 100 mm, at least 200 mm, or at least 300 mm. In some embodiments, a needle of the disclosure has a diameter of at most 0.5 mm, at most 1 mm, at most 10 mm, at most 20 mm, at most 30 mm, at most 40 mm, at most 50 mm, at most 100 mm, at most 200 mm, at most 300 mm, or at most 400 mm.

A method disclosed herein can comprise using an extruder to pass a material through a needle and onto a substrate. In some embodiments, multiple extruders deposit one or more materials onto a substrate. For example, multiple extruders can deposit material simultaneously, sequentially, or via a predefined sequence. In some embodiments, deposition from one or more extruders is controlled in real time. In some embodiments, printing is performed with 1, 2, 3, 4, 5, 6, 7, 8, 9, 10, 11, 12, 13, 14, 15, or more extruders.

The temperature at which an extruder operates can be controlled. In some embodiments, an extruder operates at a temperature of about 25° C. to about 200° C. In some embodiments, an extruder operates at a temperature of about 25° C. to about 37° C., about 25° C. to about 50° C., about 25° C. to about 75° C., about 25° C. to about 100° C., about 25° C. to about 150° C., about 25° C. to about 200° C., about 27° C. to about 37° C., about 27° C. to about 50° C.), about 27° C. to about 75° C., about 27° C. to about 100° C.), about 27° C. to about 150° C.), about 27° C. to about 200° C., about 37° C. to about 50° C., about 37° C. to about 75° C. about 37° C. to about 100° C., about 37° C. to about 150° C., about 37° C. to about 200° C., about 50° C. to about 75° C., about 50° C. to about 100° C., about 50° C. to about 150° C., about 50° C. to about 200° C., about 75° C. to about 100° C. about 75° C. to about 150° C., about 75° C. to about 200° C., about 100° C. to about 150° C., about 100° C. to about 200° C., or about 150° C. to about 200° C. In some embodiments, an extruder operates at a temperature of about 25° C. about 27° C., about 37° C., about 50° C.), about 65° C., about 75° C., about 100° C., about 150° C., or about 200° C. In some embodiments, an extruder operates at a temperature of at least about 25° C., at least about 27° C., at least about 37° C., at least about 50° C., at least about 75° C., at least about 100° C., or at least about 150° C. In some embodiments, an extruder operates at a temperature of at most about 25° C., at most about 37° C., at most about 50° C., at most about 75° C., at most about 100° C., at most about 150° C., or at most about 200° C.

In some embodiments, pressurized air is used to move a material through an extruder. The air pressure of an extruder can be controlled. In some embodiments, an extruder operates at an air pressure of about 600 kPa to about 800 kPa. In some embodiments, an extruder operates at an air pressure of about 600 kPa to about 625 kPa, about 600 kPa to about 650) kPa, about 600 kPa to about 675 kPa, about 600 kPa to about 700 kPa, about 600 kPa to about 725 kPa, about 600 kPa to about 750 kPa, about 600 kPa to about 775 kPa, about 600) kPa to about 800 kPa, about 625 kPa to about 650 kPa, about 625 kPa to about 675 kPa, about 625 kPa to about 700 kPa, about 625 kPa to about 725 kPa, about 625 kPa to about 750) kPa, about 625 kPa to about 775 kPa, about 625 kPa to about 800 kPa, about 650) kPa to about 675 kPa, about 650 kPa to about 700 kPa, about 650 kPa to about 725 kPa, about 650) kPa to about 750 kPa, about 650) kPa to about 775 kPa, about 650 kPa to about 800 kPa, about 675 kPa to about 700 kPa, about 675 kPa to about 725 kPa, about 675 kPa to about 750) kPa, about 675 kPa to about 775 kPa, about 675 kPa to about 800 kPa, about 700 kPa to about 725 kPa, about 700) kPa to about 750 kPa, about 700 kPa to about 775 kPa, about 700) kPa to about 800) kPa, about 725 kPa to about 750) kPa, about 725 kPa to about 775 kPa, about 725 kPa to about 800 kPa, about 750 kPa to about 775 kPa, about 750) kPa to about 800 kPa, or about 775 kPa to about 800 kPa. In some embodiments, an extruder operates at an air pressure of about 600 kPa, about 625 kPa, about 650 kPa, about 675 kPa, about 689.5 kPa, about 700 kPa, about 717.1 kPa, about 725 kPa, about 750) kPa, about 775 kPa, or about 800 kPa. In some embodiments, an extruder operates at an air pressure of at least about 600 kPa, at least about 625 kPa, at least about 650 kPa, at least about 675 kPa, at least about 700 kPa, at least about 725 kPa, at least about 750 kPa, or at least about 775 kPa. In some embodiments, an extruder operates at an air pressure of at most about 625 kPa, at most about 650 kPa, at most about 675 kPa, at most about 700 kPa, at most about 725 kPa, at most about 750 kPa, at most about 775 kPa, or at most about 800 kPa.

In some embodiments, an extruder operates at an air pressure of about 60 pounds per square inch (psi) to about 120 psi. In some embodiments, an extruder operates at an air pressure of about 87 psi to about 90.6 psi, about 87 psi to about 94.3 psi, about 87 psi to about 97.9 psi, about 87 psi to about 101.5 psi, about 87 psi to about 105.2 psi, about 87 psi to about 108.8 psi, about 87 psi to about 112.4 psi, about 87 psi to about 116 psi, about 90.6 psi to about 94.3 psi, about 90.6 psi to about 97.9 psi, about 90.6 psi to about 101.5 psi, about 90.6 psi to about 105.2 psi, about 90.6 psi to about 108.8 psi, about 90.6 psi to about 112.4 psi, about 90.6 psi to about 116 psi, about 94.3 psi to about 97.9 psi, about 94.3 psi to about 101.5 psi, about 94.3 psi to about 105.2 psi, about 94.3 psi to about 108.8 psi, about 94.3 psi to about 112.4 psi, about 94.3 psi to about 116 psi, about 97.9 psi to about 101.5 psi, about 97.9 psi to about 105.2 psi, about 97.9 psi to about 108.8 psi, about 97.9 psi to about 112.4 psi, about 97.9 psi to about 116 psi, about 101.5 psi to about 105.2 psi, about 101.5 psi to about 108.8 psi, about 101.5 psi to about 112.4 psi, about 101.5 psi to about 116 psi, about 105.2 psi to about 108.8 psi, about 105.2 psi to about 112.4 psi, about 105.2 psi to about 116 psi, about 108.8 psi to about 112.4 psi, about 108.8 psi to about 116 psi, or about 112.4 psi to about 116 psi. In some embodiments, an extruder operates at an air pressure of about 60 psi, about 87 psi, about 90.6 psi, about 94.3 psi, about 97.9 psi, about 100 psi, about 101.5 psi, about 104 psi, about 105.2 psi, about 108.8 psi, about 112.4 psi, about 116 psi, or about 120 psi. In some embodiments, an extruder operates at an air pressure of at least about 60 psi, at least about 87 psi, at least about 90.6 psi, at least about 94.3 psi, at least about 97.9 psi, at least about 101.5 psi, at least about 105.2 psi, at least about 108.8 psi, or at least about 112.4 psi. In some embodiments, an extruder operates at an air pressure of at most about 90.6 psi, at most about 94.3 psi, at most about 97.9 psi, at most about 101.5 psi, at most about 105.2 psi, at most about 108.8 psi, at most about 112.4 psi, at most about 116 psi, or at most about 120) psi.

A method of the disclosure can comprise printing a material at various linear extrusion speeds. In some embodiments, material is deposited at a linear extrusion speed of about 8 mm/s to about 800 mm/s. In some embodiments, material is deposited at a linear extrusion speed of about 100 mm/s to about 150 mm/s, about 100 mm/s to about 200 mm/s, about 100 mm/s to about 250) mm/s, about 100 mm/s to about 300 mm/s, about 100 mm/s to about 350 mm/s, about 100 mm/s to about 400 mm/s, about 100 mm/s to about 450 mm/s, about 100 mm/s to about 500 mm/s, about 100 mm/s to about 600 mm/s, about 100 mm/s to about 700 mm/s, about 100 mm/s to about 800 mm/s, about 150 mm/s to about 200 mm/s, about 150) mm/s to about 250) mm/s, about 150) mm/s to about 300 mm/s, about 150 mm/s to about 350) mm/s, about 150) mm/s to about 400) mm/s, about 150 mm/s to about 450) mm/s, about 150 mm/s to about 500 mm/s, about 150 mm/s to about 600 mm/s, about 150 mm/s to about 700) mm/s, about 150) mm/s to about 800 mm/s, about 200 mm/s to about 250) mm/s, about 200 mm/s to about 300 mm/s, about 200 mm/s to about 350 mm/s, about 200 mm/s to about 400) mm/s, about 200 mm/s to about 450) mm/s, about 200 mm/s to about 500 mm/s, about 200 mm/s to about 600 mm/s, about 200 mm/s to about 700 mm/s, about 200 mm/s to about 800 mm/s, about 250 mm/s to about 300 mm/s, about 250 mm/s to about 350 mm/s, about 250) mm/s to about 400 mm/s, about 250) mm/s to about 450 mm/s, about 250 mm/s to about 500) mm/s, about 250) mm/s to about 600 mm/s, about 250) mm/s to about 700 mm/s, about 250) mm/s to about 800 mm/s, about 300 mm/s to about 350 mm/s, about 300 mm/s to about 400 mm/s, about 300 mm/s to about 450) mm/s, about 300 mm/s to about 500 mm/s, about 300 mm/s to about 600 mm/s, about 300) mm/s to about 700 mm/s, about 300) mm/s to about 800 mm/s, about 350) mm/s to about 400 mm/s, about 350) mm/s to about 450) mm/s, about 350) mm/s to about 500 mm/s, about 350) mm/s to about 600 mm/s, about 350) mm/s to about 700 mm/s, about 350) mm/s to about 800 mm/s, about 400 mm/s to about 450) mm/s, about 400 mm/s to about 500 mm/s, about 400 mm/s to about 600 mm/s, about 400 mm/s to about 700) mm/s, about 400) mm/s to about 800 mm/s, about 450) mm/s to about 500 mm/s, about 450) mm/s to about 600 mm/s, about 450) mm/s to about 700 mm/s, about 450 mm/s to about 800 mm/s, about 500 mm/s to about 600 mm/s, about 500 mm/s to about 700 mm/s, about 500 mm/s to about 800 mm/s, about 600 mm/s to about 700 mm/s, about 600 mm/s to about 800 mm/s, or about 700) mm/s to about 800 mm/s. In some embodiments, material is deposited at a linear extrusion speed of about 8 mm/s, about 10 mm/s, about 100 mm/s, about 150 mm/s, about 200 mm/s, about 250 mm/s, about 300 mm/s, about 350) mm/s, about 400) mm/s, about 450) mm/s, about 500) mm/s, about 600 mm/s, about 700 mm/s, or about 800) mm/s. In some embodiments, material is deposited at a linear extrusion speed of at least about 8 mm/s, at least about 100 mm/s, at least about 150 mm/s, at least about 200 mm/s, at least about 250 mm/s, at least about 300 mm/s, at least about 350 mm/s, at least about 400) mm/s, at least about 450) mm/s, at least about 500 mm/s, at least about 600 mm/s, or at least about 700 mm/s. In some embodiments, material is deposited at a linear extrusion speed of at most about 150) mm/s, at most about 200 mm/s, at most about 250) mm/s, at most about 300) mm/s, at most about 350) mm/s, at most about 400 mm/s, at most about 450) mm/s, at most about 500 mm/s, at most about 600 mm/s, at most about 700 mm/s, or at most about 800) mm/s.

A method of the disclosure can comprise printing a material at various volumetric speeds. In some embodiments, the printing occurs with a volumetric speed of about 1 μL/s to about 100 μL/s. In some embodiments, the printing occurs with a volumetric speed of about 1 μL/s to about 5 μL/s, about 1 μL/s to about 10 μL/s, about 1 μL/s to about 15 μL/s, about 1 μL/s to about 20 μL/s, about 1 μL/s to about 25 μL/s, about 1 μL/s to about 50 μL/s, about 1 μL/s to about 100 μL/s, about 5 μL/s to about 10 μL/s, about 5 μL/s to about 15 μL/s, about 5 μL/s to about 20 μL/s, about 5 μL/s to about 25 μL/s, about 5 μL/s to about 50 μL/s, about 5 μL/s to about 100 μL/s, about 10 μL/s to about 15 μL/s, about 10 μL/s to about 20 μL/s, about 10 μL/s to about 25 μL/s, about 10 μL/s to about 50 μL/s, about 10 μL/s to about 100 μL/s, about 15 μL/s to about 20 μL/s, about 15 μL/s to about 25 μL/s, about 15 L/s to about 50 μL/s, about 15 μL/s to about 100 μL/s, about 20 μL/s to about 25 μL/s, about 20 μL/s to about 50 μL/s, about 20 μL/s to about 100 μL/s, about 25 μL/s to about 50 μL/s, about 25 μL/s to about 100 μL/s, or about 50 μL/s to about 100 μL/s. In some embodiments, the printing occurs with a volumetric speed of about 1 μL/s, about 5 μL/s, about 10 μL/s, about 15 μL/s, about 20 μL/s, about 25 μL/s, about 50 μL/s, or about 100 μL/s. In some embodiments, the printing occurs with a volumetric speed of at least about 1 μL/s, at least about 5 μL/s, at least about 10 μL/s, at least about 15 μL/s, at least about 20 μL/s, at least about 25 μL/s, or at least about 50 μL/s. In some embodiments, the printing occurs with a volumetric speed of at most about 5 μL/s, at most about 10 μL/s, at most about 15 μL/s, at most about 20 μL/s, at most about 25 μL/s, at most about 50 μL/s, or at most about 100 μL/s.

A method of the disclosure can comprise controlling the deposition of materials (e.g. polymers) with a degree of resolution. In some embodiments, a method disclosed herein can control material deposition with a resolution of about 0.01 mm to about 1 mm. In some embodiments, a method disclosed herein can control material deposition with a resolution of about 0.01 mm to about 0.05 mm, about 0.01 mm to about 0.1 mm, about 0.01 mm to about 0.2 mm, about 0.01 mm to about 0.3 mm, about 0.01 mm to about 0.4 mm, about 0.01 mm to about 0.5 mm, about 0.01 mm to about 1 mm, about 0.05 mm to about 0.1 mm, about 0.05 mm to about 0.2 mm, about 0.05 mm to about 0.3 mm, about 0.05 mm to about 0.4 mm, about 0.05 mm to about 0.5 mm, about 0.05 mm to about 1 mm, about 0.1 mm to about 0.2 mm, about 0.1 mm to about 0.3 mm, about 0.1 mm to about 0.4 mm, about 0.1 mm to about 0.5 mm, about 0.1 mm to about 1 mm, about 0.2 mm to about 0.3 mm, about 0.2 mm to about 0.4 mm, about 0.2 mm to about 0.5 mm, about 0.2 mm to about 1 mm, about 0.3 mm to about 0.4 mm, about 0.3 mm to about 0.5 mm, about 0.3 mm to about 1 mm, about 0.4 mm to about 0.5 mm, about 0.4 mm to about 1 mm, or about 0.5 mm to about 1 mm. In some embodiments, a method disclosed herein can control material deposition with a resolution of about 0.01 mm, about 0.05 mm, about 0.1 mm, about 0.2 mm, about 0.3 mm, about 0.4 mm, about 0.5 mm, or about 1 mm. In some embodiments, a method disclosed herein can control material deposition with a resolution of at least about 0.01 mm, at least about 0.05 mm, at least about 0.1 mm, at least about 0.2 mm, at least about 0.3 mm, at least about 0.4 mm, or at least about 0.5 mm. In some embodiments, a method disclosed herein can control material deposition with a resolution of at most about 0.05 mm, at most about 0.1 mm, at most about 0.2 mm, at most about 0.3 mm, at most about 0.4 mm, at most about 0.5 mm, or at most about 1 mm.

### Computer System

Bioprinting parameters such as, for example, deposition speed, extruder pressure, extruder temperature, extruder deposition patterns, the location of deposition, layer thickness, and the material deposited can be controlled by a computer system. In some embodiments, the computer system comprises a processor, a memory device, an operating system, and a software module for monitoring or operating the extruder. In some embodiments, the computer system comprises a digital processing device and includes one or more hardware central processing units (CPU). In further embodiments, the computer system includes an operating system configured to perform executable instructions. In some embodiments, the operating system is software, including programs and data, which manages the device's hardware and provides services for execution of applications. Suitable server operating systems include, by way of non-limiting examples. FreeBSD, OpenBSD, NetBSD®, Linux, Apple® Mac OS X Server®, Oracle®; Solaris®, Windows Server®, and Novell®; NetWare®. Suitable personal computer operating systems include, by way of non-limiting examples. Microsoft® Windows®, Apple® Mac OS X®, UNIX®, and UNIX-like operating systems such as GNU/Linux®. In some embodiments, the operating system is provided by cloud computing. In some embodiments a mobile smart phone operating system is used. Non-limiting examples of mobile smart phone operating systems include Nokia® Symbian® OS, Apple® iOS®, Research In Motion® BlackBerry OS®, Google Android®, Microsoft® Windows Phone® OS, Microsoft® Windows Mobile® OS, Linux, and Palm® WebOS. In some embodiments, the computer system includes a storage and/or memory device. In some embodiments, the storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the device is a storage device including, by way of non-limiting examples, CD-ROMs, DVDs, flash memory devices, magnetic disk drives, magnetic tapes drives, optical disk drives, and cloud computing-based storage. In some embodiments, the storage and/or memory device is a combination of devices such as those disclosed herein.

In some embodiments, the computer systems described herein include user interfaces. In further embodiments, the user interfaces include graphic user interfaces (GUIs), such as a Repetier-Host graphical user interface. In some embodiments, the user interfaces are interactive and present a user with menus and options for interacting with the computer systems and delivery systems described herein. In some embodiments, the computer system includes a display screen to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In some embodiments, an OLED display is a passive-matrix OLED (PMOLED) or active-matrix OLED (AMOLED) display. In some embodiments, the display is a plasma display. In some embodiments, the display is a video projector. In some embodiments, the display is a combination of displays such as those disclosed herein. In some embodiments, the device includes an input device to receive information from a user. In some embodiments, the input device is a keyboard. In some embodiments, the input device is a key pad. In some embodiments, the input device is the display screen, which is a touch screen or a multi-touch screen. In some embodiments, the input device is a microphone to capture voice or other sound input. In some embodiments, the systems, and software modules disclosed herein are intranet-based. In some embodiments, the systems and software modules are Internet-based. In some embodiments, the computer systems and software modules are World Wide Web-based. In some embodiments, the computer systems and software modules are cloud computing-based. In some embodiments, the computer systems and software modules are based on data storage devices including, by way of non-limiting examples, CD-ROMs, DVDs, flash memory devices, RAM (e.g., DRAM, SRAM, etc.), ROM (e.g., PROM, EPROM, EEPROM, etc.), magnetic tape drives, magnetic disk drives, optical disk drives, magneto-optical drives, solid-state drives, and combinations thereof.

### Methods of Use.

The biomaterials described herein can provide for the controlled and prolonged release of one or more therapeutic agents in situ at a target site in the body of a subject in need of treatment of a disease, disorder, or condition treatable by the one or more therapeutic agents. Release of the one or more therapeutic agents from the implanted article can occur through several mechanisms, including but not limited to diffusion through the polymer material, by transport through fluid-filled pores or channels in the polymer material, and through degradation of the polymer material.

Several structural features of a biomaterial disclosed herein can be adapted to modify the release of therapeutic agent(s) from the biomaterial. These structural features include but are not limited to the composition of the polymer material, the density and/or porosity of the polymer material, the sub-structure of the unit biomaterials (e.g. in a layered biomaterial) formed by the defined pattern of geometric elements, including the size, shape, and the number and arrangement of filled and open geometric elements.

The macro three-dimensional configuration of a biomaterial of the disclosure can also be adapted to modify release of therapeutic agent(s) and/or to focus release to a particular portion or region of the article. For example, a biomaterial can be folded or rolled for insertion into a target site. Several unit biomaterials can also be formed or printed into stacked layers having a desired orientation, including a staggered configuration. In some embodiments, the article can be coated with a coating. In some embodiments, the coating prevents a burst release upon placement of the biomaterial in situ. For example, the biomaterial can be coated with the same drug using a dip-coat method which is a standard method used in medicinal research and drug development. Dip coating of an article can be achieved by dipping the biomaterial into a polymer-drug solution and then drying the biomaterial to create a thin, uniform coating. Alternatively, a spray can be used to coat the biomaterial, which can allow for direct spraying of micro-droplets of a therapeutic agent onto the biomaterial itself. In some embodiments, a combination of the above can be used to coat the biomaterial using a hybrid method.

In some embodiments, a biomaterial releases one or more therapeutic agents over a period of time from about 1 day to about 1 week, about 1 week to about 1 month, about 1 month to about 2 months, about 2 months to about 6 months, about 6 months to about 12 months, about 12 months to about 24 months, about 24 months to about 42 months, about 24 months to about 54 months, or from about 24 months to about 60 months. In some embodiments, a biomaterial releases a therapeutic agent or agents over a period of time of about 24 months, about 30 months, about 36 months, about 42 months, about 54 months, or about 60) months. In some embodiments, a biomaterial releases a therapeutic agent or agents over a period of time of at least about 24 months, at least about 30 months, at least about 36 months, at least about 42 months, at least about 54 months, or at least about 60 months. In some embodiments, a biomaterial releases a therapeutic agent or agents over a period of time of at most about 24 months, at most about 30 months, at most about 36 months, at most about 42 months, at most about 54 months, or at most about 60 months.

Non-limiting examples of a subject include a human, primate, mouse, rat, dog, cat, cow, horse, goat, camel, sheep or a pig. In some embodiments, methods disclosed herein are methods of treating a subject in need thereof.

In some embodiments, a therapeutically effective amount of the one or more therapeutic agents can delivered to the in situ target site over a period of time, through implantation of a biomaterial of the disclosure.

Methods disclosed herein can treat conditions by, for example, alleviating, reducing, or reducing the likelihood of one or more symptoms or complications of a disease or disorder. In some embodiments, a method disclosed herein reduces the likelihood of a disease or disorder occurring in a subject. For example, in the context of cancer, treating the cancer can include slowing the growth of the cancer, slowing or preventing the occurrence of metastases, or further metastases, and promoting regression of one or more tumors in the subject being treated.

In some embodiments, the biomaterial is for use in releasing a drug over a period of, for example, about 3 weeks, about 4 weeks, about 30 days, or about 5 weeks. Within the period, an amount of drug can be released from the biomaterial, for example, about 40% (e.g., +/−10%), 50% (e.g., +/−10%), 55% (e.g., +/−10%), 60% (e.g., +/−10%), 65% (e.g., +/−10%), 70% (e.g., +/−10%), 75% (e.g., +/−10%), 80% (e.g., +/−10%), or 85% (e.g., +/−10%). For example, approximately 75% of a 20 mg dose of 5-FU can be released, delivering a therapeutic dose of 15 mg.

In some embodiments, the biomaterial releases about 40% (e.g., +/−10%), about 50% (e.g., +/−10%), about 55% (e.g., +/−10%), about 60% (e.g., +/−10%), about 65% (e.g., +/−10%), about 70% (e.g., +/−10%), about 75% (e.g., +/−10%), about 80% (e.g., +/−10%), or about 85% (e.g., +/−10%) of a drug over a period of three weeks. In some embodiments, the biomaterial releases about 40% (e.g., +/−10%), about 50% (e.g., +/−10%), about 55% (e.g., +/−10%), about 60% (e.g., +/−10%), about 65% (e.g., +/−10%), about 70% (e.g., +/−10%), about 75% (e.g., +/−10%), about 80% (e.g., +/−10%), or about 85% (e.g., +/−10%) of a drug over a period of four weeks. In some embodiments, the biomaterial releases about 40% (e.g., +/−10%), about 50% (e.g., +/−10%), about 55% (e.g., +/−10%), about 60% (e.g., +/−10%), about 65% (e.g., +/−10%), about 70% (e.g., +/−10%), about 75% (e.g., +/−10%), about 80% (e.g., +/−10%), or about 85% (e.g., +/−10%) of a drug over a period of five weeks. In some embodiments, the biomaterial releases about 40% (e.g., +/−10%), about 50% (e.g., +/−10%), about 55% (e.g., +/−10%), about 60% (e.g., +/−10%), about 65% (e.g., +/−10%), about 70% (e.g., +/−10%), about 75% (e.g., +/−10%), about 80% (e.g., +/−10%), or about 85% (e.g., +/−10%) of a drug over a period of 30 days.

In some embodiments, the biomaterial releases at least about 40% (e.g., +/−10%), at least about 50% (e.g., +/−10%), at least about 55% (e.g., +/−10%), at least about 60% (e.g., +/−10%), at least about 65% (e.g., +/−10%), at least about 70% (e.g., +/−10%), at least about 75% (e.g., +/−10%), at least about 80% (e.g., +/−10%), or at least about 85% (e.g., +/−10%) of a drug over a period of three weeks. In some embodiments, the biomaterial releases at least about 40% (e.g., +/−10%), at least about 50% (e.g., +/−10%), at least about 55% (e.g., +/−10%), at least about 60% (e.g., +/−10%), at least about 65% (e.g., +/−10%), at least about 70% (e.g., +/−10%), at least about 75% (e.g., +/−10%), at least about 80% (e.g., +/−10%), or at least about 85% (e.g., +/−10%) of a drug over a period of four weeks. In some embodiments, the biomaterial releases at least about 40% (e.g., +/−10%), at least about 50% (e.g., +/−10%), at least about 55% (e.g., +/−10%), at least about 60% (e.g., +/−10%), at least about 65% (e.g., +/−10%), at least about 70% (e.g., +/−10%), at least about 75% (e.g., +/−10%), at least about 80% (e.g., +/−10%), or at least about 85% (e.g., +/−10%) of a drug over a period of five weeks. In some embodiments, the biomaterial releases at least about 40% (e.g., +/−10%), at least about 50% (e.g., +/−10%), at least about 55% (e.g., +/−10%), at least about 60% (e.g., +/−10%), at least about 65% (e.g., +/−10%), at least about 70% (e.g., +/−10%), at least about 75% (e.g., +/−10%), at least about 80% (e.g., +/−10%), or at least about 85% (e.g., +/−10%) of a drug over a period of 30 days.

In some embodiments, a single dose is administered. For example, the one or more biomaterials are administered, and optionally removed, e.g., four weeks after implant. In some embodiments, repeat doses are administered. For example, the one or more biomaterials are administered, and optionally removed (e.g., four weeks after implant), then one or more biomaterials are administered a second time (e.g., about 30 days after the first dosage). In some embodiments, repeat doses are administered, for example, about every four weeks (e.g., +/−5 days), about every 30 days (e.g., +/−5 days), about every 45 days (e.g., +/−5 days), or about every 60 days (e.g., +/−5 days).

In some embodiments, a biomaterial comprises a dose of drug disclosed herein (e.g., an active pharmaceutical ingredient (API), such as 5-FU), that is about 10 mg, about 15 mg, about 20 mg, about 25 mg, about 30 mg, about 40 mg, about 60 mg, about 80 mg, about 100 mg, about 120 mg, about 140 mg, about 160 mg, or about 180 mg. In some embodiments, a biomaterial comprises a dose of drug that is about 10-180 mg, about 20-180 g, about 20-100 mg, about 10-60 mg, about 20-60 mg, about 20-40 mg or about 15-30 mg. In some embodiments, a biomaterial comprises a dose of drug that is at least about 10 mg, at least about 15 mg, at least about 20 mg, at least about 25 mg, at least about 30 mg, at least about 40 mg, at least about 60 mg, at least about 80 mg, at least about 100 mg, at least about 120 mg, at least about 140 mg, at least about 160 mg, or at least about 180 mg. In some embodiments, a biomaterial comprises a dose of drug that is at most about 10 mg, at most about 15 mg, at most about 20 mg, at most about 25 mg, at most about 30 mg, at most about 40 mg, at most about 60 mg, at most about 80 mg, at most about 100 mg, at most about 120 mg, at most about 140 mg, at most about 160 mg, or at most about 180 mg. In some embodiments, the biomaterial comprises the dose in one biomaterial unit (e.g., chip) disclosed herein. In some embodiments, the dose is divided between two, three, four, or five biomaterial units (e.g., chips). The multiple units can be separate or can be joined (e.g., sutured) together.

A biomaterial disclosed herein can limit systemic exposure to a therapeutic agent (e.g., drug). In some embodiments, a level of a drug in circulation (e.g., blood or plasma) of a subject with a biomaterial locally applied is at most 25%, at most 20%, at most 15%, at most 10%, at most 5%, or at most 1% of a level in a subject administered the therapeutic agent by a systemic route (e.g., intravenously). A biomaterial disclosed herein can limit systemic exposure to a therapeutic agent (e.g., drug). In some embodiments, a level of a drug in circulation (e.g., blood or plasma) of a subject with a biomaterial locally applied is at most 50%, at most 25%, at most 20%, at most 15%, at most 10%, at most 5%, or at most 1% of a level in a subject administered the therapeutic agent topically.

### Methods of Treating Cancer.

The present disclosure provides methods of treating cancer in a subject in need thereof, the methods comprising implanting a biomaterial of the disclosure into a target site of the subject. In some embodiments, the target site is a portion of an organ, hard tissue, soft tissue, or lymph node. In some embodiments, the target site is a solid tumor or portion thereof. The surgical article can be loaded with an amount of one or more therapeutic agents effective to provide a therapeutic dose of the one or more agents to the target site in situ for a period of time ranging from weeks, to months, to years, as described supra.

In some embodiments, the subject in need of treatment is a human patient diagnosed with a cancer, for example, colorectal cancer, anal cancer, esophageal cancer, gastric cancer, breast cancer, skin cancer, bone cancer, prostate cancer, liver cancer, lung cancer, brain cancer, cancer of the larynx, cancer of the gall bladder, pancreatic cancer, rectal cancer, parathyroid cancer, thyroid cancer, adrenal cancer, neural tissue cancer, head and neck cancer, colon cancer, stomach cancer, cancer of the bronchi, renal cancer, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, cervical dysplasia and in situ carcinoma, neuroblastoma, retinoblastoma, soft tissue sarcoma, malignant carcinoid, mycosis fungoide, rhabdomyosarcoma, Kaposi's sarcoma, osteogenic and other sarcoma, renal cell tumor, polycythemia vera, adenocarcinoma, glioblastoma multiforma, leukemias, lymphomas, malignant melanomas, epidermoid carcinomas, carcinomas, sarcomas, hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, and nodular regenerative hyperplasia.

In some embodiments, the cancer is metastatic. In some embodiments, the cancer is non-metastatic. In some embodiments, the cancer is an advanced local cancer. In some embodiments, the cancer is a recurrent locally advanced cancer.

In embodiments, a biomaterial disclosed herein can be used to treat the subject is a having a malignant cancer or late-stage cancer. In some embodiments, the subject in need of treatment can also be one that is non-responsive or refractory to a currently available therapy, or to the standard of care therapy for the disease, disorder, or condition being treated, such as the cancer.

In some embodiments, a biomaterial disclosed herein can be used to treat a colorectal cancer, for example, a colon cancer, a rectal cancer, or a bowel cancer, a gastrointestinal malignancy, or any cancer that developed in the colon or rectum. In some embodiments, a biomaterial for treating colon cancer comprises a therapeutic agent indicated for the treatment of colon cancer. In some embodiments, the biomaterial comprises a therapeutic agent selected from one or more of gemcitabine (Gemzar), raltitrexed (Tomudex™) oxaliplatin (Eloxatin™), regorafenib, irinotecan (Camptostar™), and 5-fluorouracil (Adrucil™). In some embodiments, the therapeutic agent is selected from capecitabine, fluorouracil, irinotecan and oxaliplatin, and combinations thereof.

In some embodiments, treating cancer according to the methods described herein leads to the elimination of a symptom or complication of the cancer being treated. Elimination of the symptom is not required. In some embodiments, the severity of the symptom is decreased. In the context of cancer, non-limiting examples of such symptoms include clinical markers of severity or progression including the degree to which a tumor secretes growth factors, degrades the extracellular matrix, becomes vascularized, loses adhesion to juxtaposed tissues, or metastasizes, as well as the number of metastases.

Treating cancer according to the methods described herein can result in a reduction in size of a tumor. A reduction in size of a tumor can also be referred to as tumor regression. In some embodiments, after treatment, tumor size is reduced by at least about 5% relative to the size of the tumor prior to treatment. In some embodiments, tumor size is reduced by at least about 10% after treatment. In some embodiments, tumor size is reduced by at least about 20% after treatment. In some embodiments, tumor size is reduced by at least about 30% after treatment. In some embodiments, tumor size is reduced by at least about 40% after treatment. In some embodiments, tumor size is reduced by at least about 50% after treatment. In some embodiments, tumor size is reduced by at least about 75% after treatment. In some embodiments, the size of a tumor can be measured as a diameter of the tumor.

Treating cancer according to the methods described herein can result in a reduction of tumor volume. In some embodiments, after treatment, tumor volume is reduced by at least about 5% relative to the size of the tumor prior to treatment. In some embodiments, tumor volume is reduced by at least about 10% after treatment. In some embodiments, tumor volume is reduced by at least about 20% after treatment. In some embodiments, tumor volume is reduced by at least about 30% after treatment. In some embodiments, tumor volume is reduced by at least about 40% after treatment. In some embodiments, tumor volume is reduced by at least about 50% after treatment. In some embodiments, tumor volume is reduced by at least about 75% after treatment. In some embodiments, tumor volume is reduced by at least about 60% to at least about 90% after treatment.

Treating cancer according to the methods described herein can result in a decrease in number of tumors. Tumor number can be reduced by, for example, at least about 5%, at least about 10%, at least about 20%, at least about 30%, at least about 40%, at least about 50%, or at least about 75% relative to the number of tumors prior to treatment. Number of tumors can be measured by any reproducible measurement. The number of tumors can be measured by counting tumors visible to the naked eye or at a specified magnification (e.g. 2×, 3×, 4×, 5×, 10×, or 50× magnification).

Treating cancer according to the methods described herein can result in a decrease in number of metastatic lesions in tissues or organs other than the primary tumor site. Metastatic lesions can be reduced by, for example, at least about 5%, at least about 10%, at least about 20%, at least about 30%, at least about 40%, at least about 50%, or at least about 75% relative to the number of metastatic lesions prior to treatment. Number of metastatic lesions can be measured by any reproducible measurement. The number of tumors can be measured by counting tumors visible to the naked eye or at a specified magnification (e.g. 2×, 3×, 4×, 5×, 10×, or 50× magnification).

Treating cancer according to the methods described herein can result in an increase in average survival time of a population of treated subjects in comparison to a population receiving carrier alone. An increase in average survival time of a population can be measured by any reproducible methods. An increase in average survival time of a population can be measured, for example, by calculating for a population the average length of survival following initiation of treatment with an active compound (e.g. implantation of a biomaterial loaded with a therapeutic agent). An increase in average survival time of a population can also be measured, for example, by calculating for a population the average length of survival following completion of a first round of treatment with an active compound. In some embodiments, the average survival time of a population is increased by at least 30 days, at least 60 days, at least 90 days, or at least 120 days.

Treating cancer according to the methods described herein can result in increase in average survival time of a population of treated subjects in comparison to a population receiving the standard of care therapy. In some embodiments, the average survival time of a population is increased by at least 30 days, at least 60 days, at least 90 days, or at least 120 days. An increase in average survival time of a population can be measured by any reproducible methods. An increase in average survival time of a population may be measured, for example, by calculating for a population the average length of survival following initiation of treatment with an active compound (e.g. implantation of a biomaterial loaded with a therapeutic agent). An increase in average survival time of a population can also be measured, for example, by calculating for a population the average length of survival following completion of a first round of treatment with an active compound.

Treating cancer according to the methods described herein can result in a decrease in the mortality rate of a population of treated subjects in comparison to a population receiving carrier alone. Treating cancer according to the methods described herein can result in a decrease in the mortality rate of a population of treated subjects in comparison to an untreated population. Treating cancer according to the methods described herein can result in a decrease in the mortality rate of a population of treated subjects in comparison to a population receiving the standard of care therapy. For example, the mortality rate can be decreased by at least about 2%, at least about 5%, at least about 10% or at least about 25%. A decrease in the mortality rate of a population of treated subjects can be measured by any reproducible methods. A decrease in the mortality rate of a population can be measured, for example, by calculating for a population the average number of disease-related deaths per unit time following initiation of treatment with an active compound. A decrease in the mortality rate of a population can also be measured, for example, by calculating for a population the average number of disease-related deaths per unit time following completion of a first round of treatment with an active compound.

Treating cancer according to the methods described herein can result in a decrease in tumor growth rate. In some embodiments, a method disclosed herein reduces tumor growth rate by at least about 5%, at least about 10%, at least about 30%, at least about 40%, at least about 50%, or at least about 75% relative to number prior to treatment Tumor growth rate can be measured by any reproducible measurement. Tumor growth rate can be measured according to a change in tumor diameter per unit time. In some embodiments, after treatment the tumor growth rate can be about zero and is determined to maintain the same size, i.e., has stopped growing.

Treating a cancer according to the methods described herein can result in a decrease in tumor regrowth. In some embodiments, treatment with a method disclosed herein results in tumor regrowth that is at most about 5%, at most about 10%, at most about 20%, at most about 30%, at most about 40%, at most about 50%, or at most about 75%. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

### Methods of Treating Pain.

The disclosure also provides methods of treating or managing pain in a subject in need thereof, the method comprising implanting a biomaterial described here into a target site of the subject. In some embodiments, the target sites are nerves, connective tissue and skeletal muscle and tissue. The biomaterial can be loaded with an amount of the one or more therapeutic agents effective to provide a therapeutically-effective amount of the one or more agents to the target site in situ for a period of time ranging from weeks, to months, to years, as described above. In some embodiments, the subject in need is a human patient in need of treatment for postsurgical pain, peripheral nerve injury, or chronic lower back pain. In some embodiments, the subject is in need of treatment or management of pain associated with osteoarthritis, diabetic peripheral neuropathy or musculoskeletal injury or trauma.

In some embodiments, for treating pain described herein, one or more therapeutic agents of a biomaterial is an opioid. Non-limiting examples of opioids include morphine, fentanyl, hydromorphone, codeine, oxycodone, hydrocodone, tramadol, methadone, alfentanil, remifentanil, and derivations thereof.

In some embodiments of the methods for treating pain described herein, the one or more therapeutic agents is dexamethasone, ondansetron, acetaminophen, a nonsteroidal anti-inflammatory drug (NSAID), gabapentin, pregabalin, capsaicin, ketamine, memantine, clonidine, dexmedetomidine, tapentadol, transdermal fentanyl, a long acting local anesthetic, a cannabinoid, or a combination of any of the foregoing.

### EXAMPLES

In some embodiments, a biomaterial (e.g., biomaterial unit) disclosed herein is described as a “chip”.

### Example 1: Biomaterial Formulation

### Overview

A biomaterial disclosed herein in this example is a 3D printed platform designed to deliver drugs locally at the tumor site. This strategy aimed to bypass the clinical side effects caused by the systemic toxicity of the chemotherapeutic drugs. The Biomaterial is organized structures of pores and drug concentrated regions. This entropy of drug distribution can provide a key opportunity of regulating the drug release rate as desired and can be tailored to deliver a therapeutic dose for a particular disease indication. The Biomaterial can be a pristine polymer and drug combination, e.g., without any additives or fillers reducing the likelihood of immune reactions or cross talking between various components. The polymer will have the drug dispersed in its matrix and will deliver the drug in a slow and sustained manner. The release of the drug will be defined with a varied (e.g., optimized) combination of polymer and drug properties.

The Biomaterial can address the unmet need for novel and platform drug delivery technologies. A conventional drug delivery system can involve diffusion of drug particles throughout the bloodstream, which reduces the bioavailability of the drug at the target sites. Moreover, the mode of intake in conventional mechanism involves significant disintegration of active ingredients or localization of effects. Targeted drug delivery is gaining traction owing toward significant demand for improvement in the drug delivery efficiency as well as the need to mitigate potential side-effects, which can occur due to drug accumulation. Targeted drug delivery technology started gaining pace with the advent of nanotechnology and biocompatible materials. Targeted drug delivery can have a high impact and adoption potential due to its ability to improve bioavailability and organ specificity. This trend can potentially lead to the adoption of techniques such as organ specific delivery, cell mimicking, guided delivery, and spatio-temporal control systems.

### BACKGROUND

This example relates to a pharmaceutical formulation for delivering a drug (e.g., 5-fluorouracil, Gemcitabine, etc.) locally using a 3D printed polymeric structure for the treatment of pancreatic cancer.

Millions of people around the world are suffering with cancer. Thousands of them are dying every day. Pancreatic cancer is one the deadliest cancers with 85% mortality rate. According to the American Cancer Society, there is an estimated new pancreatic cancer case of 60,430 in 2021 alone [Cancer Facts & Figures 2021]. The symptoms appear at later stages of cancer where the cancer reaches to the third or fourth stage by then in the patients. The survival chances are very minimal to none. The current standard care of treatment is removing the tumor by a clinical procedure called ‘Pancreaticoduodenectomy’ or Whipple's procedure. During operation, a part of the pancreas head, duodenum, bile duct and gallbladder will be removed to prevent any resection of the tumor. The five year survival rate is 27.4% [Improved Survival Following Pancreaticoduodenectomy to Treat Adenocarcinoma of the Pancreas The Influence of Operative Blood Loss]. However, the surgery is not an option for most of the patients diagnosed with pancreatic cancer.

Chemotherapy is another route of treatment for pancreatic cancer patients. Chemotherapy is administered either before surgery (neoadjuvant chemotherapy) to reduce tumor size so that it can be removed or after surgery (adjuvant chemotherapy) to prevent any growth of leftover tumor cells or tumor resection. Chemotherapy will also be administered to patients when surgery is no longer an option. A antineoplastic drug, such as 5-fluorouracil. Gemcitabine (2′. 2′-difluoro 2′ deoxycytidine, dFdC), or Gemcitabine Hydrochloride, can be a treatment modality administered intravenously. For example, a standard clinical dose of Gemcitabine is 1000 mg/m² of patient infused by IV over a period of 30 min. This administration can be done every week for 7 weeks, one week rest and then dosage on days 1, 8, and 15 of 28-day cycles. The common side effects of this treatment can include nausea, vomiting, rash, fever, liver transaminases elevation, flu-like symptoms. Myelosuppression, pulmonary toxicities and peripheral edema [Gemcitabine-Induced Pulmonary Toxicity: A Case Report of Pulmonary Veno-Occlusive Disease]. Recently, a drug combination. For example, a chemotherapy regimen comprising leucovorin calcium. 5-fluorouracil, irinotecan hydrochloride, and oxaliplatin was clinically used for pancreatic cancer treatment. Even though the survival rate is higher compared to surgery and Gemcitabine treatment, there are severe side effects reported for the chemotherapy regimen including Hair loss, Redness, pain or peeling of palms and soles, Rash, increased risk of sunburn, itching. Severe diarrhea, nausea, vomiting, constipation, loss of appetite, weight loss. Difficulty swallowing. Sores in mouth. Heartburn. Infection (especially when white blood cell count is low). Anemia which may require a blood transfusion. Bruising, bleeding, Headache, Tiredness, weakness, dizziness, Numbness, tingling or pain, “pins and needles” of the hands, feet, arms and legs. Tingling or a loss of feeling in your hands, feet, nose, or tightness in throat or jaw: or difficulty swallowing or breathing which may be made worse by exposure to cold. Cough, shortness of breath. Fever, and pain. Irrespective of the type of chemotherapy administered, the side effects due to the systemic toxicity of these drugs can be very prevalent in pancreatic cancer patients. The rapid availability of drugs in the bloodstream can be associated with spread throughout much of the body and action of the drug on normal cells, while only a fraction of the drug reaches the cancer cells. The unwanted effect of a drug on healthy cells can be a major reason for several severe side-effects in patients. An objective of the drug delivery treatment strategy can be to deliver therapeutic doses that are effectively available at the disease site. Hence, biomaterials disclosed herein can facilitate a sustained release of drugs (e.g., 5-fluorouracil. Gemcitabine, etc.) locally at the pancreatic tumor site reducing the availability of the drug to healthy cells.

Technology disclosed herein offers a joint solution to the problems of surgery and chemotherapy and can be delivered in parallel with standard of care surgery. Innovations in component materials as well as the ultrastructure achieved through 3D printing have resulted in a product prototype much more capable of stable release over long timescales than prior iterations, which can allow desirable effects to be achieved with a single implantation. By localizing the effects of chemotherapy, the biomaterial offers an alternative to direct surgical resection in the delicate areas surrounding the pancreas. We expect this will extend the number of patients who can benefit from surgery as well as increase the overall success of surgeries primarily by preventing local recurrence. Overall, sustained & localized release properties of the biomaterial can enhance both surgical and chemotherapeutic outcomes for PC. By reducing chemotherapy side effects & the need for in-hospital chemotherapy administration, healthcare costs can also be decreased.

The drug delivery treatment strategy involves the incorporation of drug into a polymeric structure which can be a nanoparticle, microparticles or a scaffold with two dimensional or three dimensional architecture that facilitates a slow, sustained release of drug in low amounts for longer periods. Polymers play a major role in defining the amount and time of the drug released from the polymer. The Food and Drug Administration approved several biodegradable and biocompatible polymers such as polylactide-co-glycolide (PLGA), polycaprolactone (PCL), polyglycolide (PGA), polylactide (PLA) which are widely used in drug delivery applications. PCL blended with PLA was approved by FDA as a safe food contact agent for packaging purposes. AQLANE Medical BV of Netherlands produced and urethral implant with the trade name “Urolon” which is composed of 70% CMC gel and 30% PCL microspheres. PCL is a soft and flexible polymer with −60° C. glass transition temperature and a melting point of 60° C. However, the melting point of this polymer varies with respect to the molecular weight or molecular number. As the molecular weight is higher, and the viscosity is higher. PCL has an average molecular weight of 3,000 to 90,000 g/mol. For this research we used PCL with molecular weights of 25,000, 37,000, 50,000, and 80,000. The molecular weight of PCL plays a crucial role in drug release and degradation rate. PCL undergoes a two-step degradation: first, hydrolytic cleavage of ester groups and second, intracellular degradation for PCI with molecular weights less than 3,000. PCL degrades faster outside the human body because of the bacterial enzymes rather than inside the human body as these enzymes are not available. The other factors include the geometry of the PCL structure and the microenvironment of the implanted site in the human body. The hydroxyl radical (OH·) could be a primary component for degradation of PCL in implantable devices. Hence it is clear that the molecular weight of PCL plays a major role in defining the release profile of the drug in delivery devices.

### Technical Strategy

The drug release from the polymer can be a key factor for the Biomaterial to be able to provide the therapeutic dose at the tumor site to facilitate tumor cell death. The release of the drug can be controlled in, for example, two ways. The first one is to change the polymer molecular weight which will influence the density and permeability properties of PCL. The second one is to vary (e.g., optimize) the amount of drug loaded in the polymer. Considering these two factors, the following strategies were planned.
Strategy I: To test the effects of molecular weight of PCL on drug release in case of uniform drug loading conditions.
Strategy II: To test the effects of molecular weight of PCL concurrent with different drug loading quantities on the drug release profile in vitro.

These two strategies result in finding an effective (e.g., optimized) formulation with suitable polymer that can release desired drug amounts.

Strategy 1: Preliminary understanding of the effect of molecular weight on drug release. A size of a polymer (e.g., a number average molar mass of the polymer, such as that of PCL) can have an effect on release profile (e.g., release rate) of a drug (e.g., 5-fluorouracil, Gemcitabine, etc.) from the biomaterial comprising the polymer, as disclosed herein.

For example, the PCL molecular weight might have an effect on the drug release profile in vitro. To evaluate this phenomenon, PCL with two different number average molar mass values (e.g., 37,000 and 50,000) were chosen to study with the anticancer drug 5-fluorouracil (5-FU) with an initial loading of 20 wt %. A sample of PCL having a number average molar mass of about 37,000 (i.e., PCL 37,000, PCL 37k, or PCL 37) mixed with 20 wt % Gemcitabine was mentioned as “PG37” and a sample of PCL having a number average molar mass of about 50,000 (i.e., PCL 50,000, PCL 50k, or PCL 50) mixed with 20 wt % Gemcitabine was mentioned as “PG50”. Generally, a sample of PCL having a number average molar mass of Z×10³ may be referred to as PCL Z×10³, PCL ZK, or PCL Z.

TABLE 1
Formulations of PCL and 5-FU

| | | | Gemcitabine |
|---|---|---|---|
| Formulation | PCL 37000 | PCL 50000 | (20 wt %) |
| P37 | * | — | — |
| P50 | — | * | — |
| PG37 | * | — | * |
| PG50 | — | * | * |

Hot-Melt Extrusion: The polymer of 1.6 g was mixed with 0.4 g of 5-fluorouracil and blended in a hot-melt extruder (HAAKE mini-CTW, Thermoscientific, USA) using the following parameters.

TABLE 2
Blending Parameters of PCL and 5-FU

| Parameter | Value |
|---|---|
| Temperature | 75° C. (PCL 37000) |
| | 85° C. (PCL 50000) |
| Blending (Rotations per minute (RPM)) | 200 |
| Screw rotations during extrusion (RPM) | 50 |
| Mixing time | 15 min |

3D printing: The blended formulation was taken into a metal syringe and 3D printed the Biomaterials in Allevi2 bioprinter (Allevi3D, 3D systems, USA) as per the manufacturer protocol.

Swelling: When polymers are introduced into an aqueous environment, water diffuses into the polymer porous structure, causing the polymer to swell, increasing in mass. The change of mass is referred to as the percentage swelling of a polymer, given by the equation

$$\% Swelling = ( \frac{{W}_{S} - {W}_{D}}{{W}_{D}} ) * 100 \qquad ( 1 )$$

where W_{D} is the dry weight of a polymer, and W_{S} is the swelled weight of a polymer, or the weight of a polymer taken at a certain time point after being allowed to swell in a liquid.

Upon printing, the dry mass of each chip was measured in a weighing balance (Mettler Toledo, USA). In a falcon tube, 25 mL of 0.1 M phosphate buffer saline solution (PBS, pH 7.4), and the chips were introduced into the buffer solution and allowed to incubate in a shaker flask at 37° C., and 250 rpm. After 24 hours, the chips were weighed. The chips were returned into the buffer solution and incubator and allowed to shake further for 7 days. The weight of the chips was taken and recorded.
1. Drug Release
2. Degradation

Strategy 2: Evaluation of polymer (e.g., PCL) and drug (e.g., 5-fluorouracil, Gemcitabine) formulations for enhanced (e.g., optimal) dosage and optimal molecular weight of polymer Biomaterials can be generated with one or more polymers (e.g., PCL) and drugs (e.g., 5-fluorouracil, Gemcitabine) at varied weight ratios to assess, e.g., manufacturing conditions, properties of the resulting biomaterials (e.g., drug release profile, stability, etc.). For example, biomaterials comprising PCL and Gemcitabine at varied weight ratios can be generated and analyzed.

TABLE 3
Formulations of PCL and Gemcitabine (wt/wt)

| | Gemcitabine | Gemcitabine | Gemcitabine | Gemcitabine |
|---|---|---|---|---|
| | 45 mg/100 | 60 mg/100 | 80 mg/100 | 125 mg/100 |
| | mg PCL | mg PCL | mg PCL | mg PCL |
| PCL 25000 | F-1 | F-2 | F-3 | F-4 |
| PCL 37000 | G-1 | G-2 | G-3 | G-4 |
| PCL 50000 | H-1 | H-2 | H-3 | H-4 |
| PCL 80000 | J-1 | J-2 | J-3 | J-4 |

### Results

An outcome from the experiments performed is that the lower molecular weight PCL of 25,000 and 37,000 were easy to mix with all different weights of PCL and are easier to print compared to 80,000 molecular weight PCL. While 50,000 molecular weight PCL is marginally easier compared to 80,000 molecular weight PCL and tougher compared to 25 and 37 K PCL formulations. Upon drug encapsulation and release, there is no clear difference in release profile between 37,000 and 50,000 molecular weights of PCL. The PCL lesser than 37,000 and greater than or equal to 50,000 molecular weight could be used to tailor the release profile as 80,000 molecular weight can be difficult to develop a formulation.

### Strategy 1:

### Swelling Study

The dry mass and swelled mass of each set of samples was taken at time points of 1 minute, 24 hours, and 7 days. The percentage swelling was calculated for both the 24-hour time point and the 7-day time point from Equation (1). The average percent swelling for each sample and time point can be seen in Table 4.

TABLE 4
Swelling percentage of Biomaterial samples

| | 24-hour | | 7-Day | |
|---|---|---|---|---|
| | Swelling | Standard | Swelling | Standard |
| Formulation | (% mean) | Deviation | (% mean) | Deviation |
| PCL60K | 0.00 | 0.000 | 0.00 | 0.000 |
| PCL60K | 0.45 | 0.003 | 0.78 | 0.007 |
| (negative control) | | | | |
| PCL37K | 2.45 | 0.025 | 3.43 | 0.033 |
| PCL37K | 0.00 | 0.016 | 2.25 | 0.030 |
| (negative control) | | | | |

As seen from Table 4, the total range of percentage swelling among all samples was 0% to 3.5%. The largest increase in mass occurred in the PCLdose2K sample, with a swelling percentage of 3.5% after 24 hours and 3.05% after 7 days. Additionally, the PG50 samples also had a higher swelling percentage as compared to the PCL samples. On average, the percentage swelling was greater after 7 days than at 24 hours (as compared by a 1.55% total average swelling on day 7 versus 1.34% average swelling after 24 hours for all samples).

### Degradation Study

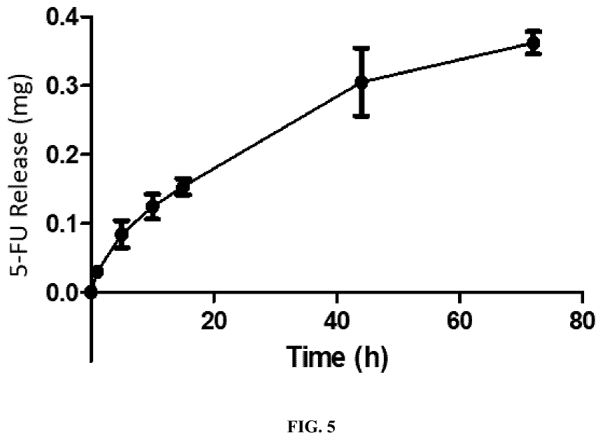

The degradation profile FIG. 6 showed around 27% degradation for PF50 and PF37 over the time of 30 days. However, there is no weight loss for P50 and P37 controls. This weight loss might be due to the release of drugs from the Biomaterials. On correlation of the weight loss with the release quantities, it is confirmation that the degradation shown by the drug loaded formulations is actually the release of drug.

### Release Study

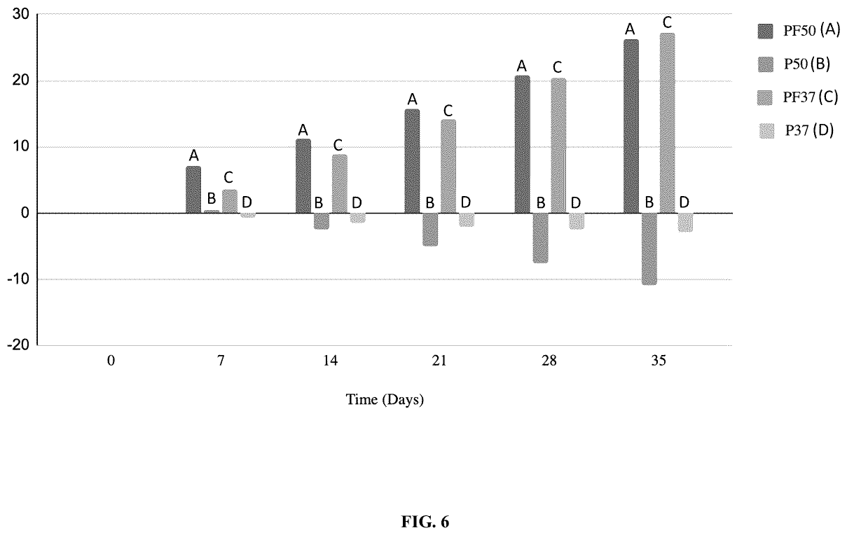

The release study for 30 days showed that both PF50 and PF37 showed sustained release (FIG. 7). PF50 showed higher drug release of 5-FU compared to PF37. The release profiles were similar between the two polymers. The higher molecular weight polymer releases increased doses of drug comparatively. This higher release could be due to high viscosity or comparative hydrophobicity of the polymer.

### Strategy 2:

The formulations of PCL with different molecular weights (25,000 [F]: 37,000 [G]: 50,000 [H] and 80,000 [J]) were mixed with different weights of Gemcitabine Hydrochloride (45, 60, 80, and 125 mg/100 mg of PCL) in a Hot-Melt Extruder to ensure uniform dispersion and mixing of drug in the polymer. The formulations with molecular weight 25,000 and 37,000 went smooth processing with all different weights of Gemcitabine under the set parameters. The formulations with molecular weight 50,000 showed some friction during mixing with 125 mg/100 mg of PCL. The process needs to repeat again to ensure uniform mixing. The formulations with molecular weight 80,000 were extruded with Gemcitabine weights of 45 and 60 mg/100 mg of PCL with repeated mixing. However, no mixing and extrusion was observed for 80,000 molecular weight PCL with Gemcitabine of 80 and 125 mg/100 mg of PCL. The results confirmed that the molecular weights 25,000:37,000; and 50,000 works well with all different weights of Gemcitabine providing a uniform mixing and extrusion.

The extruded formulations were 3D printed using the optimized print parameters of the PCL alone of different molecular weights with no Gemcitabine. However, some adjustments in temperature, pressure, and layer height were done for PCL with higher molecular weights loaded with higher amounts of Gemcitabine. The lower molecular weight PCL of 25,000 printed all the formulations with different weights of Gemcitabine at higher print speeds and lower pressures while the higher molecular weight PCL of 80,000 was very difficult to print and the formulations greater than 45 mg/100 mg PCL weren't printed at all in the 3D printer. For these 80K PCL-Gemcitabine formulations the temperature increased to 120° C., and the printed chips showed color change indicating some thermal degradation of the polymer.

### Formulation

A formulation of the biomaterial as disclosed herein can comprise one or more polymers (e.g., PCL) and a drug substance (e.g., 5-fluorouracil, gemcitabine), e.g., with the polymer molecular weights (e.g., number average molar mass) and/or the active pharmaceutical ingredient (API) ratio selected to allow controlled release of the drug substance post placement (e.g., over 4 weeks). The components can be homogeneously combined and aseptically 3D printed.

An illustrative biomaterial can comprise or consist of the following formulation:

The impermeable layer that exists within the Biomaterial is constructed from PCL 50,000 with no drug. The permeable layers are PCL 37,000 with choice of drug & PCL 50,000 with choice of drug. Various suitable compounds can be included within this formulation.

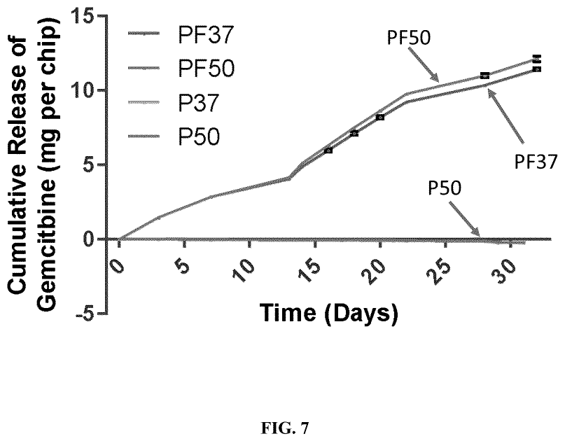

Additional (or alternative) embodiments of the biomaterial as disclosed herein are in FIG. 8, wherein the darker shading can refer to loading of one or more therapeutic agents (e.g., 5-fluorouracil, gemcitabine).

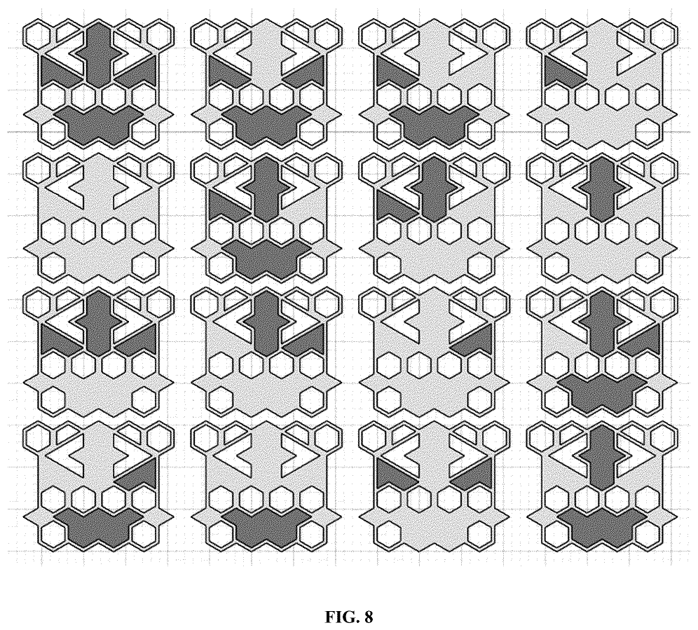

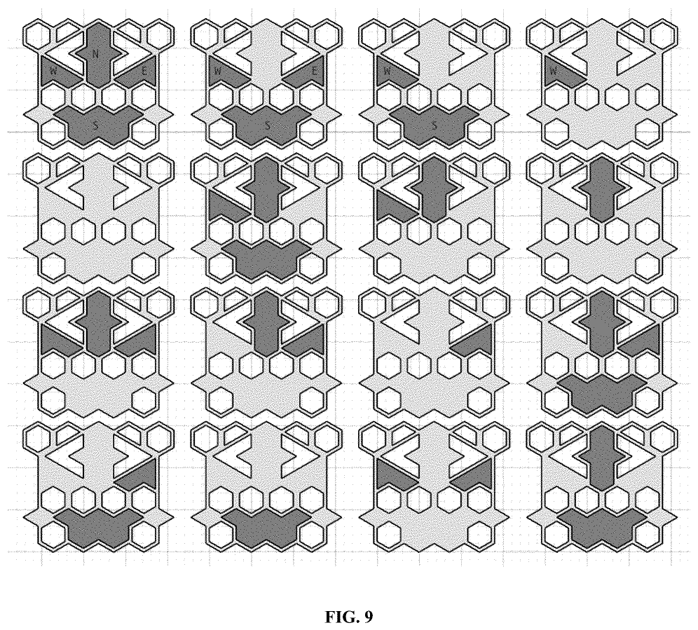

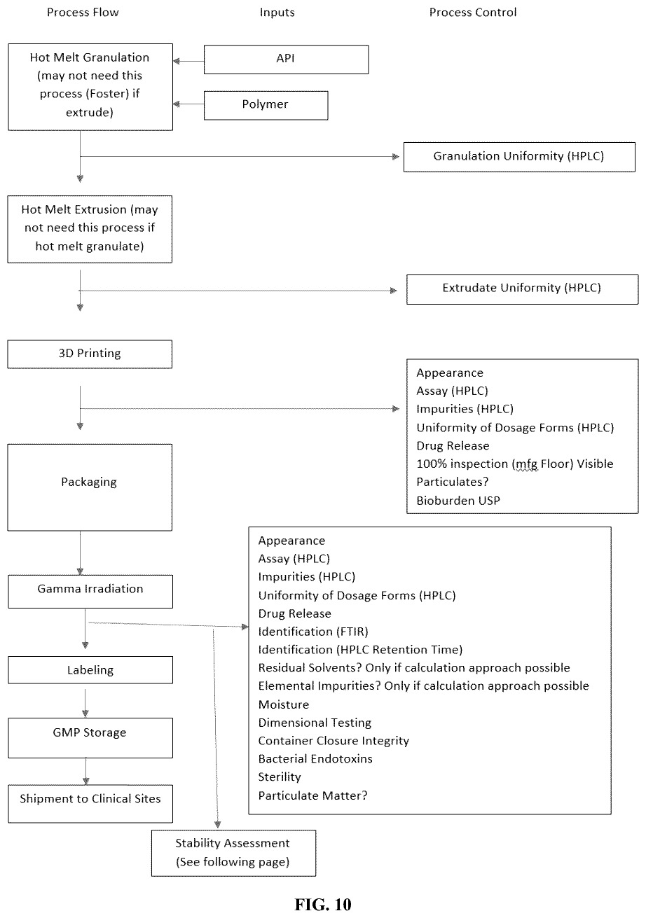

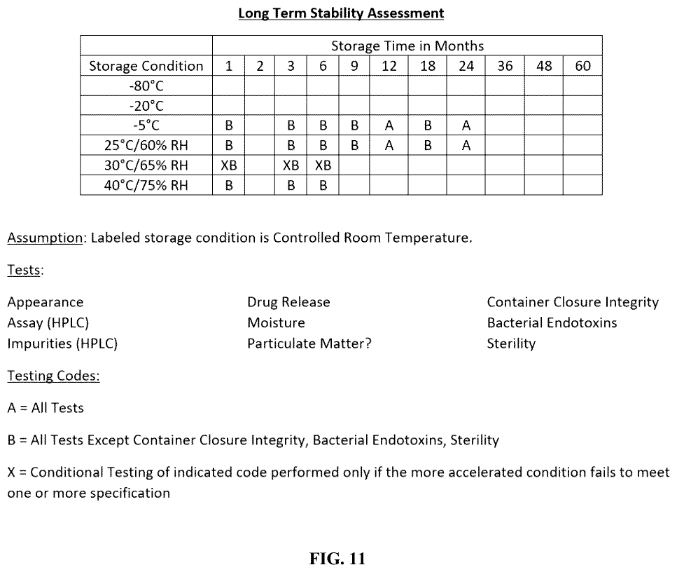

In some embodiments, as shown in FIG. 9, the biomaterial as disclosed herein can comprise a plurality of portions (e.g., N, S, W, E, etc.) capable of comprising drugs (e.g., 5-fluorouracil, gemcitabine). In some cases, the drugs can be loaded to one or more portions of the plurality of portions of the biomaterial. In some cases, the drugs can be loaded to two or more portions of the plurality of portions of the biomaterial. The two or more portions can comprise a first portion and a second portion, and the first portion and the second portion can be characterized by having (1) different polymers. (2) same polymers but having different number average molecular weight. (3) different drugs. (4) same drug but having different loading amount, and/or (5) different weight ratios between the polymer and the drug within each portion.

In some examples, a biomaterial can be loaded with a drug (e.g., 5-fluorouracil) in four different portions: N, S, E, and W. The portions N and S can have substantially the same parameters: (i) comprising a high molecular weight polymer (e.g., PCL having a number average molecular weight of about 50,000 Dalton), and (ii) comprising substantially the same ratio between such polymer and the loaded drug (e.g., a weight ratio of about 1:0.73 (polymer:drug), such as about 19 milligrams PCL and about 14 milligrams drug). In addition, the portions E and W can have substantially the same parameters: (i) comprising a low molecular weight polymer (e.g., PCL having a number average molecular weight of about 37,000 Dalton), and (ii) comprising substantially the same ratio between such polymer and the loaded drug (e.g., a weight ratio of about 1:0.50 (polymer:drug), such as about 12 milligrams PCL and about 6 milligrams drug). The biomaterial can comprise an additional region (e.g., frame) that surrounds at least a portion of one or more of the four different portions, and the additional region can be loaded with a drug. The additional region can be characterized by (i) comprising a high molecular weight polymer (e.g., PCL having a number average molecular weight of about 50,000 Dalton, and 45 milligrams of such polymer), and (ii) substantially free of the drug.

In some embodiments, a biomaterial can be loaded with a drug (e.g., 5-fluorouracil) in one or more of four different portions: N, S, E, and W. The portions N and S can have substantially the same parameters: (i) comprising a high molecular weight polymer (e.g., PCL having a number average molecular weight of about 50,000 Dalton), and (ii) comprising substantially the same ratio between such polymer and the loaded drug (e.g., a weight ratio of about 2:0.73 (polymer:drug), such as about 40 milligrams PCL and about 14 milligrams drug). In addition, the portions E and W can have substantially the same parameters: (i) comprising a low molecular weight polymer (e.g., PCL having a number average molecular weight of about 37,000 Dalton), and (ii) comprising substantially the same ratio between such polymer and the loaded drug (e.g., a weight ratio of about 2:0.50 (polymer:drug), such as about 24 milligrams PCL and about 6 milligrams drug). The biomaterial can comprise an additional region (e.g., frame) that surrounds at least a portion of one or more of the four different portions, and the additional region can be loaded with a drug. The additional region can be characterized by (i) comprising a high molecular weight polymer (e.g., PCL having a number average molecular weight of about 50,000 Dalton, and 90 milligrams of such polymer), and (ii) substantially free of the drug.

TABLE 5

| | Permeable section | Permeable section 2 |
|---|---|---|
| Impermeable | (darker section | (darker section |
| layer (yellow) | on the top) | on the bottom) |
| 50,000 | 37,000 | 50,000 |
| 50,000 | 50,000 | 50,000 |
| 50,000 | 25,000 | 37,000 |
| 50,000 | 37,000 | 25,000 |
| 37,000 | 50,000 | 37,000 |
| 37,000 | 37,000 | 50,000 |
| 50,000 | 50,000 | 50,000 |
| 50,000 | 50,000 | 37,000 |

If the drug molecule is above (>>) weight then a lower molecular weight polymer can be utilized for the permeable layer (ability to have more pores)

If the drug molecule is too small (<<dKA) weight then a higher molecular weight polymer can be utilized for the permeable layer

For the impermeable layer, 50,000 MW will be the standard weight.

In some embodiments, additional aspects of the biomaterial as disclosed herein can be changed for drug loadability, including, but not limited to, size of a polymer of the biomaterial, size of the therapeutic agent, polarity (or charge) of the biomaterial, polarity (or charge) of the therapeutic agent, and/or printability (e.g., ability to withstand manufacturing process & temperature) of the polymer of the biomaterial and/or of the therapeutic agent.

### Example 2: Overview of Dosage Form, Manufacturing, and Administration

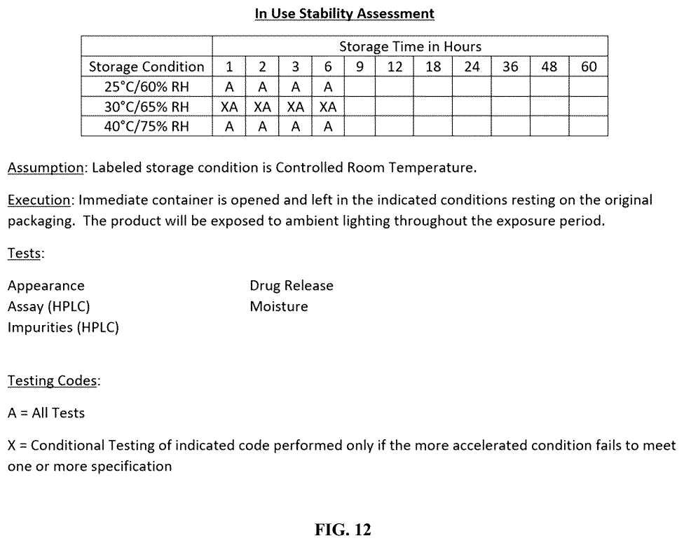

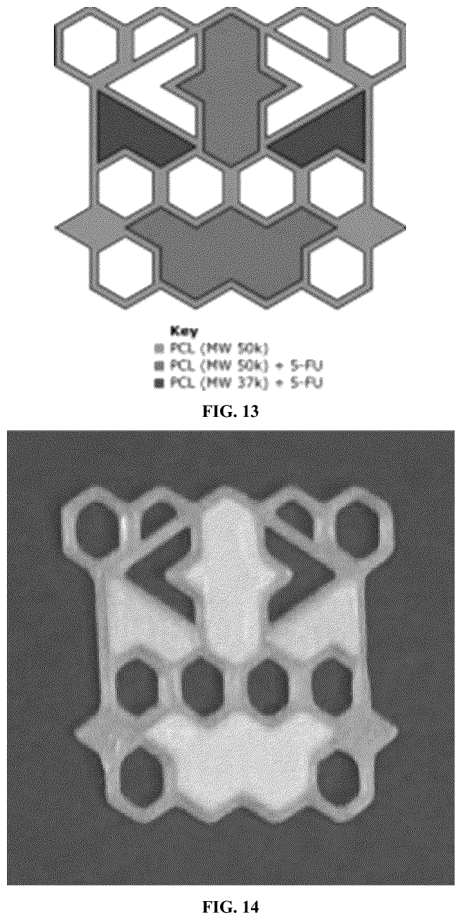

Dosage form: Biomaterial-5-FU is a flexible extended-release biodegradable dosage form composed of fluorouracil encapsulated in a pure polymer matrix (polycaprolactone, PCL). Each Biomaterial-5-FU (20 mg) contains 20 mg of fluorouracil. A 2-dimensional image of the chip design is shown in FIG. 13 and a photograph in FIG. 14.

### Approximate Dimensions of Biomaterial-5-FU:

L×W×H: 20 mm×20 mm×0.8 mm
Mass is approximately 200 mg

Brief Overview of Manufacturing Process: Biomaterials are produced using an Additive Manufacturing (AM) multi-extruder printing system that uses three containers with either biodegradable polymer or polymer-drug mixtures. The printer deposits the materials listed in Table 5 in a very precisely controlled spatial organization to form a structure which contains 4 drug compartments. For the Biomaterial-5-FU, two compartments contain PCL-37,000 kDa MW with active pharmaceutical ingredient (API: see FIG. 13, darkest shading) and two contain PCL-50,000 kDa MW with API (see FIG. 13, central parts with medium shading shading), and 50,000 kDa MW supporting material alone (see FIG. 13, light shading).

TABLE 6
Distribution of 5-FU in Regions of Biomaterial-5-FU

Amount per unit (Chip)

| Section - Compartment- Segment | 5-FU | PCL |
|---|---|---|
| Polycaprolactone + 5-Fluorouracil | 8 mg | 32 mg |
| (MW: 37,000) | | |
| Polycaprolactone + 5-Fluorouracil | 12 mg | 40 mg |
| (MW: 50,000) | | |
| Polycaprolactone alone | 0 | 40 mg |
| (MW: 50,000) | | |
| Total | 20 mg | |

Route of Administration: Biomaterial is implanted/placed adjacent to tumors. More than one chip can be implanted by connecting one Biomaterial to another through suturing the corner hexagonal structures together. The Biomaterial can be administered by a method that utilizes endoscopy, colonoscopy, or traditional open surgery.

### Example 3: Preclinical Studies

The initial safety profile of Biomaterial-5-FU was established in a healthy rat model (BSPN001). The objective of the study was to determine gross and local toxicity of Biomaterial when implanted subcutaneously. No major toxicology or safety concerns were seen. No gross toxicity seen in the five major organs. Mild local inflammatory response in line with foreign body implants at the site was seen in all 3 animals.

Efficacy was tested in murine model (BSPN02). CT26 colorectal cell lines in a murine model were chosen to conduct toxicity and safety studies in-vivo. The safety profile of Biomaterial-5-FU was compared with other control groups, and five aspects were evaluated as follows: (i) change in the body weight over time, (ii) organ weight and the ratio of organ to body weight, (iii) Hematoxylin-Eosin (H & E) staining for histological changes, (iv) a hepatic function panel, and (v) blood chemistry to assess impact on bone marrow. For H & E staining, five major organs (liver, heart, spleen, lungs, and kidneys) were collected from the mice at each time point. Biomaterial did not have any effects on vital organ functions, including cardiovascular, respiratory and central nervous system. All organs exhibited no signs of local or systemic toxicity with the application of Biomaterial. Biomaterial demonstrated a superior safety profile (p<0.05) in comparison to intravenous bolus of 5-FU in short-term toxicity as shown by blood chemistry. The study showed no significant toxic effects, enzyme levels of all treatment groups were not statistically different from the untreated control group. Short-term whole blood toxicity, 3 days post-treatment, displayed differences in the blood counts between the groups (Intravenous vs Biomaterial). Data showed that IV 5-FU by itself had a toxic effect, as indicated by the reduction in the absolute numbers of white blood cells. This toxic effect was not observed in mice treated with Biomaterial-5-FU. Subpopulations of white blood cells exhibited signs of potential toxicity for animals treated with systemic (intravenous) 5-FU: a reduction in the number of monocytes and other leukocytes, indicating bone marrow suppression. This toxic effect was not observed in mice treated with Biomaterial-5-FU.

Treatment of Biomaterial in CT26 colorectal mice demonstrated a delay in tumor growth and statistically significant reduction of tumor burden. A pre-clinical non-GLP efficacy study showed sustained release of local 5-FU onto the subcutaneous CT26 colorectal carcinoma murine model as well. In comparison to a blank Biomaterial or no intervention, the mice treated with Biomaterial-5-FU exhibited the smallest tumor weight increases, significantly smaller tumor burden than the untreated and blank chip (P<0.05). Mice in the groups receiving 5-FU (Biomaterial-5-FU and 5-FU unconfigured chip) exhibited slower tumor growth compared to the mice in groups that did not receive 5-FU (untreated group and blank Biomaterial group). This was evidenced by smaller increases in the tumor weight. By day 19 Biomaterial-5-FU demonstrated a strong anti-tumor effect compared to other interventions. Dose escalation toxicities were tested in large porcine model (BSPN003) with up to 5 Biomaterial units placed within the peritoneal cavity. In a 3-day study, the safety profile of Biomaterial-5-FU was determined by (i) change in the body weight over time, (ii) change in bowel movements or displacement of Biomaterial, (iii) gross pathological changes, (iv) Hematoxylin-Eosin (H & E) staining for histological changes, and (v) blood chemistry at the following timepoints: pre-dose, 8 hours, 16 hours, 24 hours, 32 hours, 40 hours, 48 hours, and 72 hours. For H & E staining, five major organs (liver, heart, spleen, lungs, and kidneys) were collected from the pig at the end of study. No organs exhibited signs of local or systemic toxicity with the application of Biomaterial. No changes in body weight over time were seen. No change in bowel movements were observed. No displacement of Biomaterial surgically applied into the intraluminal aspect of the bowel occurred. No blood chemistry tests exhibited signs of systemic toxicity or dose-associated systemic toxicity. No abnormalities were seen on gross inspection of the five major organs (liver, heart, spleen, lungs, and kidneys). No systemic adverse effects, nor significant local toxicity, were seen in the highest dose (5 Biomaterial units). No meaningful toxicological, neurological or histological findings were observed in the in-vivo studies except for localized inflammation (<1 mm) at the site of implantation on visual inspection at the time of necropsy. These studies established non clinical proof of principle. Collectively these preclinical studies support the concept that Biomaterial is a safe and efficient local delivery system, with minimal dose-limiting systemic adverse effects.

TABLE 7

| Study | | | |
|---|---|---|---|
| Number | Species & (n=) | Overall Objective | Primary Outcome |
| BSPN001 | Healthy Rat - | Overall Safety with | No major safety concerns, no gross |
| | Subcutaneous | Gross and Local | toxicity seen in the five major organs. |
| | n = 4 | Toxicity | Local inflammatory response in line |
| | | | with foreign body implants at the site. |
| BSPN002 | CT26 colorectal | Investigate Gross & | No gross or local toxicities seen, no |
| | carcinoma | Local Toxicity, | systemic toxicities seen. Observed |
| | murine model | Systemic Toxicity in | superior safety profile compared to IV |
| | n = 37 | comparison to IV 5-FU | 5-FU. |
| BSPN003 | Healthy pigs - | Investigate Gross and | Localized inflammatory response |
| | Direct Organ | Local Toxicity | observed at the site of implant upon |
| | Contact - Dose | | tissue collection. No gross toxicities in |
| | Escalation | | five organs. Local histopathology |
| | n = 3 | | pending. |

### Example 4: GLP Toxicity Study

A GLP toxicology study is conducted on a biomaterial disclosed herein utilizing Yucatan mini-pigs to assess local tolerance of fluorouracil and establish if an acute immune response induces rejection, and/or toxicity. Survival rate, acute mortality and/or impacts overall survival/morbidity are observed for the duration of 28-days. Objectives of GLP tox study are to evaluate gross organ toxicity, systemic exposure, systemic toxicity, local exposure of Biomaterial-5-FU, and local toxicity of Biomaterial-5-FU. The primary endpoints include gross observations at necropsy, toxicology, histological analysis, and pharmacokinetics analysis.

Blood samples for toxicokinetic evaluation will be collected from all animals prior to dosing/surgery, then again at 8, 16, 24, 32, 40 and 48 hours post-surgery, and on Days 3, 4, 5, 6, 7, 14, 21, and 28. A total of 324 samples will be processed for analysis for levels of fluorouracil (API) analysis by high sensitivity Liquid Chromatography-Mass Spectrometry (LC-MS). This will further serve to show that the Biomaterial-5-FU will release the API in a slow sustained manner over the course of the 28 days, and that dose dumping does not occur. Pharmacokinetics will be assessed through tissue analysis of 5-FU at the site of insertion (and surrounding tissues, including lymph nodes) at Days 7, 14, 21 and 28. This analysis will be conducted by high sensitivity LC-MS.

Evidence of local toxicity and without a significant broader regional or systemic effect can be observed. Samples will be collected for clinical pathology prior to surgery, on Days 3, 7, 14, 21 and 28 post-surgery. Images will be taken of gross tissue, pre/post Biomaterial placement and pre/post Biomaterial surgical removal. Samples will be analyzed for serum chemistry (including amylase), hematology (including reticulocytes) and coagulation parameters (PT, APTT and fibrinogen). Complete necropsy of all animals with selected tissue retention and weighing: heart, kidneys, liver, pancreas, spleen, stomach, thymus and surrounding tissue around Biomaterial placement. H & E staining and histopathological evaluation will be conducted.

Operation—For all groups, the Biomaterial is implanted directly onto the intrarectal wall through endoscopy/colonoscopy of the animal. In the event this is not possible due to the anatomy of the animal, Biomaterial/5 FU will be implanted onto the intrarectal wall through an open procedure.

Outcome measures—The measures of success and acceptance criteria can include mortality of the pigs. The measures of success and acceptance criteria can include drug tolerance of the pigs. Histology samples at the site of the biomaterial placement will be evaluated to look for necrosis or immune response immediately at the site of application. Localized drug release will be evaluated to establish pharmacokinetics in the animal. Success criteria can include all pigs remaining alive until the sacrifice point, exhibiting no signs of infection, immune response or rejection, and remaining in generally well conditions.

Criteria for early termination can include severe adverse clinical signs indicative of necrosis or organ failure, animals being moribund or in severe pain or distress.

### Example 5: Clinical Study

Human subjects (patients) are selected with progressive, locally advanced rectal and anal cancers.

Initial evaluation and feasibility occurs at the time of implantation through visual inspection and the Principal Investigator's judgment. To ensure continued placement of Biomaterial, the a pelvic X-ray is done at week 2 of the 4-week maximum application duration in Cohort 1. The final evaluation of the placement will occur at the end of the 4-week treatment cycle upon removal of Biomaterial. The criteria for repeat application will depend on the clinical results from Part I and the clinician's discretion.

Within current guidelines (ASCO, 2022), there are multiple timepoints in the treatment of this patient population that allows for safety evaluations. Generally, locally advanced rectal cancers received total neoadjuvant treatment (TNT-chemo, followed by radiation or radiation followed by chemo). The goal of TNT is to reduce tumor burden and potentially downstage the tumor prior to surgical resection. Initial evaluation of this patient population includes physical examination, imaging through MRI, followed by endoscopic evaluation. If radiation is the initial treatment, another MRI, PE and endoscope is proposed after therapy (e.g., for short-term 5 day radiation, one month later time point: for long-term 5 week radiation, may wait 6-8 weeks for eval). In the case of short-term radiation (5-day course), the follow-up assessments are one month later. With long-course radiation (5-week course), the follow-up is 6-8 weeks later. Regardless of duration, most patients can undergo systemic chemotherapy one month after radiation course. The interim between radiation and chemotherapy can be the window of opportunity for use of Biomaterial-5-FU. Incidence of dose limiting toxicity (DLT) is assessed and compared to DLT associated with systemic delivery, e.g., during the first four weeks of treatment. In the case of Grade 1, 2, 3 Local Adverse Events, treatment can be continued. In the case of Grade 4 Local Adverse Events, however, the biomaterial will be removed. DLTs can include the following:

TABLE 8

| | |
|---|---|
| Severe Grade 3 | Mucosal ulceration (>1 cm), mucosal perforation without |
| | obstruction, severe rectal pain (6-10 on the Likert scale) requiring |
| | intervention, severe |
| | rectal bleeding requiring intervention |
| Potentially Life- | Mucosal perforation with obstruction, excessive bleeding leading to |
| Threatening Grade | systemic response, rectal obstruction, bowel obstruction, local |
| 4 | infectionincluding perianal abscesses or fistulas related to |
| | Biomaterial-5-FU |
| | placement |

Plasma pK analysis of 5-FU is done via LCMS at the set follow-up time points on days 1, 2, 14, and 28, to test whether dose dumping is occurring and whether there is a sustained/minimal systemic presence of 5-FU. If any sever adverse events (SAEs) are reported the Plasma pK will be evaluated at that time. Additionally, pK histology analysis by LCMS will be conducted on the excised tumors (either scheduled or resultant from SAE). LC MS can potentially detect the picograms per mL that could be observed systematically from a local application of 5-FU.

The objective of the PK sampling schedule can be to quantify systemic concentrations and tumoral/tissue concentrations of 5-FU and its associated metabolites. While the systemic concentrations of 5-FU can be low or not detectable, using a suitably sensitive bioanalytical assay, the systemic PK sampling schedule can capture the initial release profile of 5-FU, ongoing release, and clearance following removal of the biomaterial-5-FU. Tumor and tissue sampling aims to establish 5-FU concentrations in the target tissue and the surrounding tissues.

Plasma pharmacokinetic analysis of 5-FU by LC-MS is done, e.g., on Days 1, 8, 15, 22, and 28 to evaluate profile release over time and systemic exposure of 5-FU.

Sampling can be done on day 0—pre-dose, followed by 1, 2, 6 and 24 hours post-insertion to evaluate initial dose dumping. Additional sampling will assess accumulation and release monitoring of plasma 5-FU at Days 8, 15, 22, 28. To characterize elimination of 5-FU from the system, post-removal pharmacokinetic sampling will be taken on Day 28 (1, 2, 6 and 24 hours post-removal).

Additional pharmacokinetic sampling can be done at the time of removal of biomaterial-5-FU through tumor and tissue biopsy. In subjects with local tolerability issues or those with unplanned removal, a tumor and surrounding tissue biopsy can be done.

Patients with known DPD deficiency can be tested prior to inclusion into the trial. If any SAEs are reported, a plasma PK sample will be taken at that time. Additionally, PK analysis by LC-MS will be conducted on the excised tumors (either scheduled or resultant from SAE).

Biomaterial contains 20 mg of 5-Fluorouracil, with an average release profile of 75% over 4 weeks, an effective dose of 15 mg+/−. The local anti-tumor effects of the biomaterial are evaluated. The dose can be escalated, e.g., until local DLTs occur.

TABLE 9
Cohort for Part 1 Dose Escalation

| | |
|---|---|
| 1 | Subjects with histologically proven progressive locally advanced rectal or anal cancer |

Cohort for Part 2 Expansion

| | |
|---|---|
| 2A | Subjects with histologically proven rectal cancer [T2N (any), T3N (any), T4N (any)] that have |
| | received long-course radiotherapy (LCCT) or short-course radiation, will get Biomaterial |
| | placement after radiation therapy |
| 2B | Subjects with histologically proven rectal cancer [T2N (any), T3N (any), T4N (any)] that have |
| | received standard chemotherapy will get Biomaterial placement prior to the long-course |
| | radiotherapy (LCCT) or short-course radiation and/or surgical excision |
| 2C | Subjects with histologically proven, stage II/III rectal cancer (T2N0, T3N0, T4N0) who are |
| | scheduled to receive definitive surgery without chemoradiation will get Biomaterial |
| | placement |
| 3 | Subjects with locally advanced, bulky (>5 cm), stage II/III colorectal tumors who are |
| | ineligible for standard of care will get Biomaterial placement |

Cohort 3 can include subjects with bulky colorectal tumors. This expansion cohort can allow further dose escalation depending on observed results.

The primary signs of effectiveness will be determined by changes in size of the tumor, number of R0 resections, incidence of DLTs (vs that of systemic chemo), improvement in symptoms from baseline, improvement in quality of life, and potentially downstaging of the tumor confirmed via imaging modalities such as MRI/CT. To confirm release rate from Biomaterial, LC-MS analysis will be performed on plasma at set intervals (see Schedule of Assessments) and upon removal of the tumor, that tissue will also be assessed for concentration of 5-FU by LC-MS.

### Example 6: Pharmacokinetics

Pharmacokinetics of a biomaterial disclosed herein is tested in large porcine model with up to 5 Biomaterial units placed within the peritoneal cavity. At the following timepoints, blood samples are taken to determine the concentration of systemic 5-FU; pre-dose, 8 hours, 16 hours, 24 hours, 32 hours, 40 hours, 48 hours, and 72 hours. Given the local nature of the product and quantity of API, systemic exposure of 5-FU can be below the level of detection, and the majority of the 5-FU concentration can be found locally, around the Biomaterial. Details of the pharmacokinetic tissue analysis is shown below.

TABLE 10

| | | | Analytical |
|---|---|---|---|
| | # of | | Measurement |
| | Biomaterial | | of 5-FU |
| | units | | concentration |
| Group | implanted | Site | in Tissue |
| 1 | 1 | Colorectal - intraluminal bowel | LC-MS |
| | | | HPLC |
| 2 | 3 | Peritoneal wall, | LC-MS |
| | | Peritoneal - liver, and | HPLC |
| | | Colorectal - serosa of bowel | |
| 3 | 5 | Peritoneal cavity (right), | LC-MS |
| | | Peritoneal cavity (left), | HPLC |
| | | Mesentery (1), | |
| | | Colorectal - serosa of bowel, and | |
| | | Colorectal - serosa of bowel | |
| | | within 2 cm of the above | |
| | | placement | |

### Example 7: In Vitro Drug Release

The release characteristics of Biomaterial were evaluated at 0.1 M PBS at 37° C. Three Biomaterial units were submerged in an aqueous environment. Biomaterial units were removed from the PBS at 15 mins, 30 mins, 1 hour (1H), 2H, 4H, 6H, 8H, 12H, 24H, 48H, 72H (Day 3), and Day 4, 5, 6, 7, 9, 12, 14, 18, 21, 28 (week 4), week 5, 6, 7, 8, 9, 10, 11, 12 (Month 3), Month 4, 5, and 6. The levels of fluorouracil in PBS were determined by HPLC. An initial burst was seen within 24 hours where 17% of the 5-FU is released. By Day 7, 50% of 5-FU has been released. By Day 28, 75% of 5-FU is released from Biomaterial-5-FU.

TABLE 11

| Methodology | HPLC |
|---|---|
| Units | 1 Biomaterial-5-FU |
| Percent of | 10% of 5-FU released in 8 H |
| 5-FU released | 13% of 5-FU released in 12 H |
| | 17% of 5-FU released in 24 H |
| | 32% of 5-FU released by 3 D |
| | 49% of 5-FU released by 7 D |
| | 65% of 5-FU released by 14 D |
| | 70% of 5-FU released by 21 D |
| | 75% of 5-FU released by 28 D |

### Example 8: Methods of Making

A drug substance is generated that comprises 5-Fluorouracil (5-FU) as an active agent (Chemical Name: 5-fluoro-1H-pyrimidine-2,4-dione; C4H3FN2O2), at a weight of 20 mg. Polycaprolactone (PCL) is used as an excipient (Chemical Name: (1,7)-Polyoxepan-2-one; (C₆H₁₀O₂)ₙ). The PCL is at a molecular weight of 37,000 (PCL-37 k) or 50,000 (PCL-50 k). The PCL is used at a weight of 32 mg (PCL-37 k) or 40 mg (PCL-50 k).

The manufacturing process is performed in a clean-room environment with open product handling. The facilities are continuously monitored and clean rooms are designed according to EU Grade D.

For a process run of approximately 100 chips, the batch formula for the 3 intermediate materials used was:

TABLE 12

| | 100 chip batch | 5FU | PCL 37k | PCL 50k |
|---|---|---|---|---|
| Chip Type | (weight, g) | (g) | (g) | (g) |
| 0.8 mm | 21 g | 2.1 g | 2.6 g | 16.3 g |
| Biomaterial-5FU | | | | |
| (20 mg) | | | | |

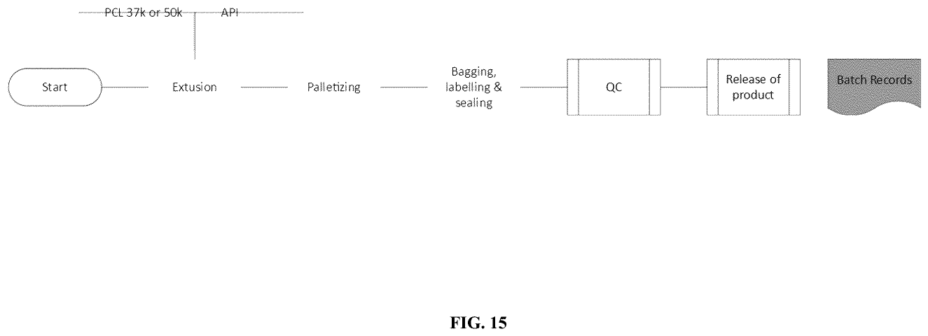

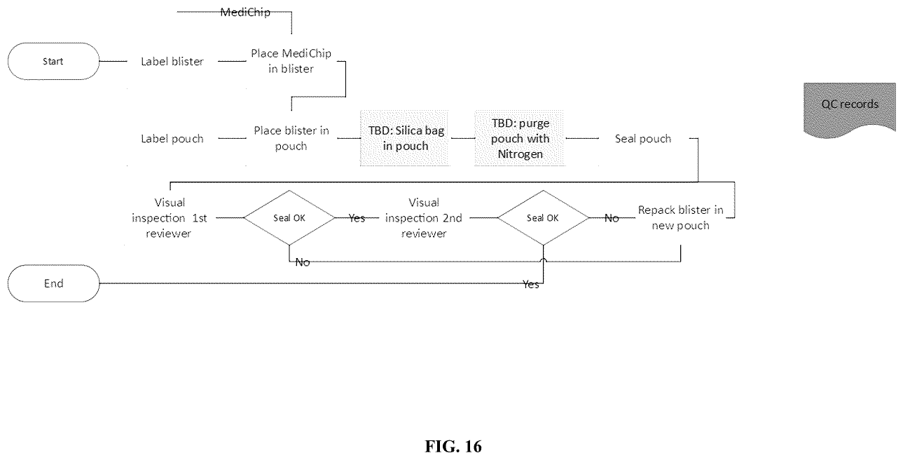

The process of making the biomaterial can be summarized as in FIG. 15.

Mixing of 5-FU with Polymer

Three biomaterials are generated: 1) PCL 37k+API: 2) PCL 50k+API; and 3) PCL 50k (alone).

The PCL (37 or 50 kDa) and 5-FU (as applicable) are mixed at a ratio of 20% drug weight in via two gravimetric feeders—(one Pharma 11 Twin Screw Feeder Mini Twin Gravimetric feeder) and oneGravimetric Roto Tube Hygienic)—designed for the hot melt extruder Pharma 11 (Thermo Fisher Scientific (Thermo Electron GmbH; Karlsruhe, Germany).

### Extrusion and Intermediate Storage

A hot melt extruder (Pharma 11 (Thermo Electron GmbH: Karlsruhe, Germany) or comparable) is used to extrude the PCL-5-FU (or PCL alone) into strands of approx. 2 mm diameter which are then wound onto cleaned transparent PET spools, using a filament spooler from Thermo Fisher with hygienic contact surfaces with FDA approved materials. Spools are placed in sealed PE or aluminum pouches until further use.
QC: Samples of the extruded material are visually inspected: Analytical parameters to be tested for each batch (experimental and clinical) include (i) Amount of 5FU in mix (wt %); and (ii) GPC (Mn, Mw, Mn/Mw).

### Biomaterial Printing

Preparation: The desired amount/range of extruded material is weighed. Each set of PCL and PCL-API composite are placed into one of the three printer extrusion chambers. Process: Biomaterial is produced via an extrusion-based additive manufacturing (AM) process wherein the three constituent ratios of PCL and API are precisely deposited in specific, three-dimensional spatial locations. This is done by placing each set of PCL and PCL-API composite into one of the three extrusion chambers and heating the chambers until the PCL reaches its glass transition temperature and is able to flow (approximately 100° C.). Once the glass transition temperature is achieved, a pneumatic system pushes the PCL and/or PCL-API composite through a stainless steel syringe tip and precisely deposits the material in pre-specified locations, building up the entire Biomaterial construct layer-by-layer. By extruding material precisely at the glass transition temperature, the extruded material transitions back to a fully solid state as soon as it is extruded out of the system (e.g., substantially upon extrusion from the system), creating very little geometric or structural variation from chip to chip.

The successful fabrication of Biomaterial through AM is achieved through a specific, validated, and pre-determined machine code that determines the motions, flow rates, and temperatures that will be achieved by the 3D printer over the entire duration of the printing process (approximately 10 minutes). The mechatronic systems controlling the 3D printer have numerous feedback loops, safety checks, and sensor systems, which serve to keep the system within a tight operating envelope with respect to material temperature, composition, spatial resolution, and mass flow rate. As such, the proper operation of these systems can be crucial to the successful manufacture of the Biomaterial and regular calibration checks can be performed using independent validation systems (in addition to the control systems built-in to the 3D printer) in order to ensure the system is consistently operating within the accepted range of values.

In some embodiments, a method of making disclosed herein comprises use of a process in which three chambers are used to print the discrete regions of biodegradable polymer or polymer-drug mixtures that characterize the Biomaterial design and allow it to have the desired biodegradation behaviors and drug release profiles. For example, three chambers (each containing PCL37-5FU, PCL50-5FU, or PCL50 alone) are heated to 105° C., the point at which the polymer or polymer-drug mixtures can flow, and pneumatic pressure is applied to drive the material out of a nozzle affixed to the stainless steel chamber. The three distinct chambers allow for the geometrically desired printing of 3 distinct materials each of which can thus be very precisely controlled in its spatial deposition.

QC: Visual inspection, Biomaterial dimension (Length, width, and thickness) and mass are measured. These measurements can indicate if the total amount of 5FU in the Chip is within specification. The amount of 5FU per chip is measured. 5FU and Polymer quality are monitored.

### Primary Packaging

The Biomaterial is placed in a clamshell blister. The blister is labelled. The blister is placed in an aluminium pouch with optionally a silica bag and optionally purged with nitrogen before sealing. The pouch is labelled. The seal is 100% visually inspected by two operators for seal quality (e.g. detecting presence of creases). If the seal is considered faulty, the Biomaterials are removed from the pouch and the blister with product is placed in a fresh labelled pouch and further processed as described herein.

### Secondary Packaging

Ten Biomaterials are packaged in a carton box (approximate dimensions—e.g. 2″×2″×3″) and boxes are packaged in a transporter (e.g., reusable, cool, cardboard). For example, ten Biomaterial units are packaged in a carton box (approximate dimensions—e.g. 7″×4″×6″) and not more than 50 carton boxes are packaged in a carton transporter. The transporter is a defined, fixed size box used during sterilization. Lesser quantities of 10-chip cartons can be transported to individual clinical sites. Transportation of the material can be done at a maximum temperature of 75 degrees Fahrenheit.

### Sterilization

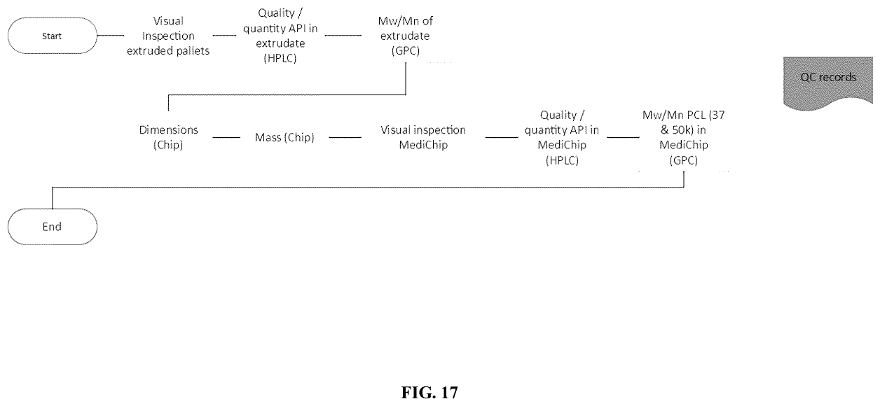

Following provisional release, boxes with Biomaterial in primary packaging are sent to the sterilizer for gamma irradiation. The transporter boxes can be individually gamma irradiated.
QC: Transporter boxes can be opened and a subset of packages of chips are evaluated for bioburden, endotoxin, and sterility. Upon return of the products, a visual inspection of the gamma dot can be done as well as the inspection of the sterilization certificate and dosage report (FIG. 17).

The biomaterial can be supplied as a sterile, solid/flexible surgical article packaged in a clamshell blister, sealed within an aluminum pouch (or, e.g., silica bag), optionally purged with nitrogen before sealing, and labeled. A label can comprise, for example, a serial or batch number, a dosage, a percent of an active drug (e.g., w/w, w/v, or v/v), and instructions for storage of the drug (e.g., 15-25° C., do not freeze).

### Example 9: Clinical Study

Cohort 1—These are the first patients in the expansion. This can include any subject regardless of previous treatment. This cohort can be used, e.g., to evaluate additional safety and efficacy in these patients who have previously been treated. These patients can have advanced cancers and have already received multiple therapies and have failed treatment. These subjects can be locally advanced cancer patients with local recurrent lesions.
Cohort 2—To isolate the effect of Biomaterial as a chemo replacement. Subjects are locally advanced cancer patients that have received long-term radiation-so these are POST-radiation treatment but have not had any chemotherapy. A primary purpose of this cohort can be to look at efficacy and safety in the treatment with post-radiation and pre-chemotherapy patients.
Cohort 3—to determine if there is an effect of Biomaterial with radiation pre-chemotherapy. Includes locally advanced cancer patients who are scheduled for short-term radiation therapy. This cohort can include subjects with smaller, localized tumors that can be easily accessed after short-term (e.g., 5-day) radiation, that have not yet received other chemotherapy.
Cohort 4—dose escalation colorectal cancer. Subjects can receive, e.g., 6-8 Biomaterial units per subject. The patient population criteria can be the same as cohort 1-any and all patients in colorectal who have had treatment. Evaluating safety of increasing dose while potentially some early clinical benefit.

### Example 10: Development of Biomaterials

Prototype biomaterials were tested with various polymer molecular weight and combinations of various parameters (printing, design, drug weights, polymer weights etc.). The formulations which yielded the most suitable release profile and suitability for manufacturing were selected. The ratio for the formulation was determined by conducting several release profile experiments using HPLC. The final formulation known as Biomaterial-5-FU then underwent several in-vitro studies to determine the chemical and mechanical properties of the final product. Encapsulation measurements, stimulated release in various pHs (pH 1, pH 5, pH 7.4, pH 8, pH 10), uniformity of dosage, tensile strength, forced degradation studies, mechanical integrity, compressive strength, release profiles post-degradation, and effects of swelling on Biomaterial-5-FU were studied. Experiments conducted included those shown in Table 13:

TABLE 13

| Experiment | Description | Objective | Results | Outcome |
|---|---|---|---|---|
| Formulations | Development of | Determine | PCL 37K and | Finalized MWs |
| | various | suitable MW of | PCL50K | of PCL for |
| | combinations of | PCL in the 3 | yielded suitable | Biomaterial |
| | PCL MW | formulations | biphasic release | |
| | | | profiles | |
| Evaluate Print | Tested various | Determine | PCL37K MW: | Finalized |
| Parameters | combinations of | suitable | PSI 100 +/− 10 | printing |
| | temperature, | printing | at 80° C. | parameters of |
| | pressure, | parameter | PCL50K MW: | PCL for final |
| | dimensions, and | combination | PSI 105 +/− 2 at | formulation of |
| | speed of printing | for PCL37K | 105° C. | Biomaterial |
| | | MW and | | |
| | | PCL50K MW | | |
| Differential | Crystallinity of | Determine if | Crystallization | Drug compound |
| Scanning | the drug on the | crystallization | of 5-FU | was active and |
| Calorimetry I | final Biomaterial | of the 5FU was | occurred over a | unaltered - no |
| | | occurring | print range of | impact on |
| | | during the | 140° C. to 240° C. | manufacturing - |
| | | process of | | current print |
| | | incorporation | | temp is 105° C. |
| | | into the | | |
| | | polymer | | |
| Differential | Assessment of | Determine if | No | Drug compound |
| Scanning | 5-FU | crystallization | crystallization | was active and |
| Calorimetry II | crystallinity in | of the 5FU | was found | unaltered |
| | pH 7.45 over 4 | occurred over | | |
| | months | the 4-month | | |
| | | duration | | |
| Differential | Assessment of | Determine if | No | Drug compound |
| Scanning | 5-FU | crystallization | crystallization | was active and |
| Calorimetry III | crystallinity in | of the 5FU | was found | unaltered |
| | pH 1 over 4 | occurred over | | |
| | months | the 4-month | | |
| | | duration | | |
| Compression | Compression | Determine the | Concentric | Concentric print |
| and Tensile | and tensile test | Ultimate | found to be | configuration |
| Test I | for concentric | Strength, | ideal fill type. | chosen |
| | fill, zig zag, and | Fracture Point, | PCL 37K | |
| | grid print | and Young's | stronger than | |
| | configurations at | Modulus for | PCL 50 which | |
| | various | each | was found by | |
| | compression | | higher ultimate | |
| | strengths on | | strengths, | |
| | newly printed | | fracture points, | |
| | Biomaterial | | and Young's | |
| | | | Moduli. | |
| Compression | Compression | Determine the | pH 1 showed | |
| and Tensile | and Tensile test | Ultimate | lower ultimate | |
| Test II | for concentric | Strength, | strengths and | |
| | fill, zig zag, and | Fracture Point, | fracture points | |
| | grid print | and Young's | | |
| | configurations | Modulus for | | |
| | on a 4-month | each | | |
| | Biomaterial | | | |
| | degraded in pH | | | |
| | 1 | | | |
| Compression | Compression | Determine the | | |
| and Tensile | test for | Ultimate | | |
| Test III | concentric fill, | Strength, | | |
| | zig zag, and grid | Fracture Point, | | |
| | configurations | and Young's | | |
| | on a 4-month | Modulus for | | |
| | degraded | each | | |
| | Biomaterial in | | | |
| | pH 7.45 | | | |
| Compression | Compression | Determine at | No cracking | Compressive |
| and Tensile | test for scaled up | what point the | was seen, only | forces do not |
| Test IV | models of the | Biomaterial | deformation | pose a large |
| | outer hexagons | outer hexagons | | threat to the |
| | of the | cracked | | suture points |
| | Biomaterial | | | |
| Compression | Tensile tests for | Determine the | Biomaterial | The Biomaterial |
| and Tensile | normal and | Ultimate | degraded in pH 1 | is able to retain |
| Test V | double height | Strength and | for 3 months | a large amount |
| | Biomaterial, as | Fracture Point | retained over | of its strength |
| | well as | for each | 90% of its | after prolonged |
| | Biomaterial | | ultimate | degradation |
| | degraded in pH | | strength | |
| | 1 | | | |
| X-Ray | Assessment of | Determine | PCL crystalline | Mechanical |
| Diffraction | PCL | mechanical | units were not | integrity of |
| (XRD) | crystallinity | integrity of | affected by the | PCL37K and |
| | | polymer | composition nor | PCL50K |
| | | through | by loading with | remained intact |
| | | crystallinity | 5-FU | |
| Scanning | Assessment of | Determine the | Tuned structures | Confirmed 5- |
| Electron | 5-FU loading | porosity of | with porosities | FU loading into |
| Microscopy | into PCL | PCL with and | allowing for | the pores of |
| (SEM) | | without drug | drug loading | PCL37K and |
| | | compound | and interactions | PCL 50K |
| | | | were observed | |
| Fourier | Encapsulation of | Determine if 5- | FTIR confirmed | Confirmed drug |
| Transformation | drug into | FU was | peaks of 5-FU | encapsulation |
| Infrared (FTIR) | polymer | encapsulated | and PCL, | into PCL37K |
| | | into PCL37K | confirming | and PCL50K |
| | | and PCL50K | encapsulation | |
| | | | of drug into | |
| | | | polymer | |
| Raman | Use lasers to | Inform about | Confirmed | Confirmed drug |
| | monitor | the state of the | peaks of 5-FU | encapsulation |
| | incorporation | drug | and PCL, | into PCL37K |
| | and specificity | | confirming | and PCL50K |
| | of the carrier | | encapsulation | |
| | system | | of drug into | |
| | | | polymer | |
| In-Vitro | Release of 5-FU | Determine the | HPLC showed >75% | Confirmation of |
| Release (IVR) | in PBS (pH | release profile | of 5-FU released | release for this |
| Profiles I | 7.45) | at conditions | in 4 weeks | formulation |
| | | found in the | | |
| | | colon | | |
| In-Vitro | Stimulated | Determine | Release after | Additional |
| Release (IVR) | release profiles | release profile | 4 weeks: | indications |
| Profiles II\| | determined by | at physiological | pH 1: 80% | identified based |
| | HPLC in various | pHs | pH 7.45: 75% | on pH release |
| | pHs - pH 1, pH | | pH 8: 80% | profiles- |
| | 7.4, pH 8 & pH | | pH 10: 64% | specifically |
| | 10 | | | potential gastric |
| | | | | and esophageal |
| | | | | applications |

### Example 11: Clinical Study

TABLE 14

| PROTOCOL TITLE | A Human Dose Escalation Study with expansion cohorts to Investigate the | |
|---|---|---|
| | Functionality, Safety, Tolerability, Pharmacokinetics, and Clinical Activity of | |
| | Biomaterial-5-Fluorouracil (5-FU) in Subjects with Progressive Locally | |
| | Advanced lower GI (e.g., rectal or anal) Cancer | |
| STUDY DESIGN | This study is divided into 2 Parts: | |
| | Part 1 is a traditional dose escalation (3 + 3) phase to establish the safety and | |
| | preliminary activity of locally inserted Biomaterial-5-FU in subjects with | |
| | locally progressive rectal or anal cancer. The starting dose for Part 1 is a single | |
| | Biomaterial carrying 20 mg of 5-FU. Eligible subjects will be enrolled in the | |
| | dosing cohorts evaluating an increasing number of inserted Biomaterial units | |
| | until a maximum tolerated dose (MTD) is established. The maximum dose for | |
| | Part 1 is not to exceed 4 Biomaterial units each carrying 20 mg 5-FU, | |
| | corresponding to a total of 80 mg 5-FU. At the end of the treatment period for | |
| | each patient (e.g., maximum 4 weeks), each inserted Biomaterial-5-FU will be | |
| | removed. | |
| | Patients will be enrolled and treated in cohorts of 3. After insertion of the | |
| | biomaterial unit(s), subjects will be followed for up to about 4 weeks and | |
| | assessed for dose-limiting toxicities (DLTs), defined as any Grade 4 Local | |
| | Adverse Events. If 0/3 DLTs occur in this cohort, then escalation to the next | |
| | dose level can proceed. If 1/3 DLT occurs, then 3 additional patients will be | |
| | treated at the current dose level. If no more than 1/6 DLT occurs, escalation to | |
| | the next dose level may proceed. If at the first dose level, more than 1/3 or more | |
| | than 1/6 DLTs occur, the study will stop or dose will be lowered. If at a higher | |
| | dose level, more than 1/3 or more than 1/6 DLTs occur, the previous dose level | |
| | is selected as the MTD. If at the highest dose level, no more than 0/3 or no more | |
| | than 1/6 DLT occurs then this dose level is considered the MTD. The MTD will | |
| | be the recommended dose for part 2. | |
| | In Part 2, the recommended dose will be given to the expansion cohorts which | |
| | are planned to further explore the safety and PK, and to obtain signals of | |
| | clinical activity of Biomaterial-5-FU in: | |
| | 1. | Subjects with early stage rectal cancer post-radiation (long-term or short- |
| | | term), pre-chemotherapy |
| | 2. | Subjects with early stage rectal cancer who are candidates for |
| | | chemotherapy, pre-radiation |
| | 3. | Subjects with early stage rectal cancer who are scheduled to receive |
| | | definitive surgery without radiation |
| | 4. | Subjects with locally advanced, bulky stage II/III colorectal tumors, who |
| | | have received or are ineligible for standard of care to further establish dose |
| | | escalation of Biomaterial |

Study investigators will review the safety data after each subject completes two
weeks on the study. Subjects can receive repeat insertions of Biomaterial-5-FU
in this study until disease progression, unacceptable toxicity, or withdrawal of
consent. Repeat insertions might be considered, e.g., every 30 days (+/−5)
depending upon tumor response and safety. Biomaterials can be reinserted at
the same dose used initially or at the next level dose (e.g., with a maximum of
four biomaterial units).

OBJECTIVES AND ENDPOINTS

| | |
|---|---|
| PRIMARY | Part 1: |
| OBJECTIVES | To determine the feasibility, safety, tolerability of Biomaterial-5-FU |
| | placement in subjects with Progressive Locally Advanced Rectal or |
| | Anal Cancer |
| | To determine a Recommended dose in subjects with Recurrent Locally |
| | Advanced Rectal or Anal Cancer |
| | Part 2: |
| | To characterize further the feasibility, safety, tolerability of |
| | Biomaterial-5-FU placement in specific expansion cohorts |
| | To explore clinical activity of Biomaterial-5-FU in specific expansion |
| | cohorts |
| PRIMARY ENDPOINTS | Part 1: |
| | Ability to place Biomaterial-5-FU at insertion site and to stay in-situ for |
| | duration of proposed therapy from physician/surgeon reports and X-ray |
| | imaging to evaluate placement, integrity, and polymer characterization of |
| | Biomaterials one week after placement and at the investigator's discretion |
| | thereafter |
| | Subject incidence of Adverse Events (AEs), Serious Adverse Events |
| | (SAEs), Treatment Emergent AEs (TEAEs) |
| | Subject incidence of Dose Limiting Toxicities (DLT): safety as the |
| | percentage of patients experiencing dose-limiting toxicities (DLTs) or |
| | serious adverse events (SAEs) using National Cancer Institute (NCI) |
| | Common Terminology Criteria for Adverse Events (CTCAE) version 4.0 |
| | (Phase I) |
| | Number of withdrawals due to AEs and changes in safety assessments (e.g. |
| | clinical laboratory parameters, vital signs, physical examination, etc.) |
| | Part 2: |
| | Continue collecting information on Biomaterial-5-FU placement and |
| | integrity, and subject incidences of AEs, SAEs, TEAEs, and DLTs with |
| | Biomaterial-5-FU placement within expansion cohorts |
| | Estimate the overall response rate (ORR) by RECIST 1.1 |
| SECONDARY | Part 1: |
| OBJECTIVES | To determine the systemic exposure to 5-FU with Biomaterial-5-FU |
| | To characterize the pharmacokinetics (PK) of 5-FU release in tumor |
| | and tissue following administration of Biomaterial-5-FU |
| | To evaluate the relationship between Biomaterial-5-FU exposure and |
| | safety/efficacy/PD responses |
| | To determine preliminary activity in subjects with Progressive Locally |
| | Advanced Rectal or Anal Cancer |
| | Part 2: |
| | To continue characterizing systemic exposure to 5-FU and the |
| | pharmacokinetics (PK) of 5-FU release in tumor and tissue following |
| | administration of Biomaterial-5-FU in specific expansion cohorts |
| SECONDARY | Part 1: |
| ENDPOINTS | PK parameters derived from plasma concentration time-data |
| | PK parameters derived from tumor and surrounding tissue (e.g., within |
| | 5 cm) concentration |
| | Number of patients with complete response (CR), partial response (PR), |
| | stable disease (SD) and progressive disease (PD) |
| | Part 2: |
| | PK parameters derived from plasma concentration time-data |
| | PK parameters derived from tumor and surrounding tissue (e.g., within |
| | 5 cm) concentration |
| | Number of patients with complete response (CR) and partial response |
| | (PR), Progressive disease (PD) and stable disease (SD) |
| | Measures of treatment efficacy depending upon individual tumor |
| | characteristics, including the number of patients with complete |
| | response (CR) and partial response (PR) as defined by Habr Gamma |
| | 2010 and other evidence of anti-tumoractivity, such as change in |
| | biopsy status (e.g. tumor cell viability or cytotoxic effects), evidence |
| | of pathological response, conversion to resectability or downstaging, |
| | R0 resection, sphincter preservation, and symptom improvement for |
| | each patient |
| EXPLORATORY | Part 1 and Part 2: |
| OBJECTIVES | To evaluate the effect of Biomaterial-5-FU on tumor histology and |
| | morphology |
| | To evaluate subjects' symptom burden during treatment with Biomaterial- |
| | 5-FU |
| | To characterize durability of response and progression free survival in each |
| | expansion cohort with reduction of tumor volume |
| | To determine tumor response rates (including downstaging) and duration of |
| | response at tumor sites in patients with advanced malignancies |
| | To explore preliminary activity in subjects with Locally Advanced Rectal or |
| | Anal Cancer |
| EXPLORATORY | Part 1 and 2: |
| ENDPOINTS | Changes in histology of tumor and tissue biopsy specimens |
| | Changes from baseline in specific biomarkers such as CEA |
| | Change from baseline in local symptom burden |
| | R0 resection rates |
| | Duration of response (DoR), Progression free survival (PFS) |
| INVESTIGATIONAL | Biomaterial-5-FU uses additive manufacturing for a localized biodegradable |
| PRODUCT | delivery of 5-FU encapsulated in polymers. The Biomaterial is flexible and can |
| | be locally applied directly target tumors in hard to reach sites. |
| SAMPLE SIZE | Approximately 70 patients (inclusive both Part 1 and 2). |
| | Part 1 will include up to 24 subjects. |
| | Part 2 will include up to 4 cohorts; each cohort will include up to 10 subjects for |
| | a total of up to 40 patients. |
| | Additional patients may be added to Part 1 and Part 2 in order to elucidate more |
| | information on the safety and activity of Biomaterial, e.g., based on safety, pK, |
| | and tumor assessments |
| DOSE JUSTIFICATION | Prior studies have shown that large doses of intraperitoneal 5-FU have potential |
| | adverse effects such as peritonitis, prolonged ileus but superior efficacy due to |
| | bioavailability of drug at the site. Approved systemic dose for 5-FU in CRC is |
| | 400 mg/m2 followed by 2400 mg/m2 to 3000 mg/m2 every 2 weeks. |
| | Based on preclinical data, plasma levels from Biomaterial are anticipated to be |
| | 1/10th topical levels. |
| | Preclinical experiments showed no appreciable systemic exposure with |
| | Biomaterial containing 20 mg 5-FU, whereas tumor reduction (−60% to −90%) |
| | was comparable to standard dose given systemically. |
| TREATMENT ROUTE | Biomaterial-5-FU is applied during anoscopic and/or endoscopic procedure and |
| AND REGIMEN | sutured peri-tumoral. Biomaterial-5-FU can also be applied during open |
| | surgery. |
| DOSE | The starting dose will be 20 mg of 5-FU within Biomaterial. Dose escalation |
| | will occur in Part 1 and dose adjustments are allowed to address tolerability and |
| | safety issues. |
| | Part 1: Dose escalation (3 + 3) with increasing numbers of Biomaterial units. |
| | Initial dose includes placement of a single 20 mg Biomaterial-5-FU. Utilizing a |
| | 3 + 3 design, Biomaterial units will be increased (2 × 20 mg [total 40 mg], 3 × 20 |
| | mg [total 60 mg], 4 × 20 mg [total 80 mg]). |
| | Part 2: Biomaterials dose for Part 2 cohorts will be determined on the totality |
| | of PK, safety (all cycles), and activity data from Part 1. The colorectal |
| | expansion cohort will determine additional dose-escalation and safety for |
| | increasing numbers of Biomaterial (e.g., for bulky colorectal tumors). |

ELIGIBILITY CRITERIA

| | |
|---|---|
| INCLUSION CRITERIA | Part 1: |
| | Cohort 1: Histologically proven Progressive Locally Advanced Rectal or Anal |
| | Cancer |
| | Have completed standard of care, chemotherapy alone, radiation alone, |
| | or chemoradiation |
| | Or are non-responsive to the standard of care |
| | Or are ineligible for the standard of care therapy |
| | Or have declined standard of care therapy |
| | Part 2: |
| | Cohort 2A: Subjects with histologically proven rectal cancer [T2N (any), T3N |
| | (any), T4N (any)] that have received long-course radiotherapy (LCCT) or short- |
| | course radiation, will then get a Biomaterial placement |
| | Have completed long-course or short-course radiation |
| | Or are non-responsive to the radiation |
| | Or are ineligible/declined standard of care therapy |
| | Cohort 2B: Subjects with histologically proven rectal cancer [T2N (any), T3N |
| | (any), T4N (any)] that have received standard chemotherapy will then get a |
| | Biomaterial placement prior to the long-course radiotherapy (LCCT) or short- |
| | course radiation and/or surgical excision |
| | Candidates for systemic chemotherapy |
| | Or are non-responsive to the chemotherapy |
| | Or are ineligible/declined standard of care therapy |
| | Cohort 2C: Subjects with histologically proven, stage II/III rectal cancer (T2N0, |
| | T3N0, T4N0) who are scheduled to receive definitive surgery without |
| | chemoradiation |
| | Candidates for Surgical Excision |
| | Or are ineligible/declined short-term or long-term radiation |
| | Cohort 3: Subjects with locally advanced, bulky (e.g., >5 cm), stage II/III |
| | colorectal tumors who are unresectable, or who are ineligible for or have |
| | completed standard of care |
| | Have completed standard of care, chemotherapy alone, radiation alone, |
| | or chemoradiation |
| | Or are non-responsive to the standard of care |
| | Or are ineligible for the standard of care |
| | Or have declined standard of care |
| | For all subjects |
| | Ability to provide informed consent |
| | Age >18 years |
| | Bioptically verified |
| | Tumor accessible for Biomaterial placement |
| | Must have at least one lesion of evaluable size; lesion must be |
| | amenable to biopsy (core or fine needle aspirate) and application of |
| | Biomaterial (>1 cm) |
| | ECOG performance status of 0-2 |
| | Life expectancy of >3 months, in the opinion of the investigator |
| | Adequate organ and marrow function as defined below: |
| | 1. Absolute neutrophil count ≥1,000/mcL |
| | 2. Platelets ≥100,000/mcL |
| | 3. Total bilirubin 1.5 × ULN≤ |
| | 4. AST(SGOT)/ALT(SGPT) ≤3 × institutional ULN |
| | 5. Creatinine ≤ institutional ULN |
| | 6. Glomerular filtration rate (GFR) ≥50 mL/min/1.73 m² |
| | Subjects with treated brain metastases are eligible if there is no |
| | evidence of progression for at least 4 weeks after CNS-directed |
| | treatment, as ascertained by clinical examination and brain imaging |
| | (MRI or CT) during the screening period. |
| | Subjects with new or progressive brain metastases (active brain |
| | metastases) or leptomeningeal disease are eligible if the treating |
| | physician determines that immediate CNS specific treatment is not |
| | required and is unlikely to be required during the study treatment |
| | period. |
| | Subjects with a prior or concurrent malignancy whose natural history or |
| | treatment does not have the potential to interfere with the safety or |
| | efficacy assessment of the Biomaterial placement should be |
| | included. |
| | Subjects with known history or current symptoms of cardiac disease, or |
| | history of treatment with cardiotoxic agents, should have a clinical risk |
| | assessment of cardiac function using the New York Heart Association |
| | Functional Classification. To be eligible for this trial, patients should be |
| | Class 2 or better. |
| PHARMACOKINETIC/ | Part 1 of this study will have extensive serial sampling for PK and PD |
| PHARMACODYNAMIC | measurements. Single safety PK blood draws may be collected for subjects with |
| ASSESSMENTS | severe adverse events or adverse events of concern. In addition, pre-treatment |
| | and post-treatment tumor tissue samples will be required for Part 1 and Part 2 |
| | (based upon the Investigator's judgment) to evaluate safety and efficacy of |
| | Biomaterial-5-FU on tumor biology. |
| SAFETY | Routine physical examinations, vital sign measurements, clinical laboratory |
| ASSESSMENTS | tests, and monitoring of adverse events. X-Ray at week 1 to ensure |
| | Biomaterial(s) remains in place, with follow-up imaging at a frequency |
| | determined by the PI. All x-ray imaging will be assessed by a single |
| | Radiologist. Biomaterial should be removed/changed at a maximum interval of |
| | 40 days, if well tolerated. Additional safety assessment may be necessary if the |
| | patient undergoes radiation or other cytotoxic treatments. |
| STATISTICAL | For part 1 a 3 + 3 design is used. With 4 dose levels this means that there will be |
| CONSIDERATIONS | maximum 24 patients. |
| | For part 2, no formal hypothesis was used to calculate sample sizes. |
| | The primary analysis will occur when target enrollment is complete and each |
| | subject either completes 3 months of study or withdraws from the study. |
| | Descriptive statistics will be provided for selected demographics, safety, PK, |
| | efficacy and biomarker data by dose, dose schedule, and time as appropriate. |

### EMBODIMENTS

### Embodiment 1

A biomaterial comprising a plurality of geometric elements and a therapeutic agent, wherein:
a first geometric element of the plurality of geometric elements comprises a first polymer and the therapeutic agent, wherein at least a portion of the first geometric element is porous;
a second geometric element of the plurality of geometric elements comprises a second polymer, wherein at least a portion of the second geometric element is substantially non-porous;
the at least the portion of the first geometric element is adjacent to the at least the portion of the second geometric element; and
the first polymer or the second polymer has a number average molar mass of greater than 6,000 Dalton (Da),
optionally wherein:
(A) the first polymer and the second polymer have a number average molar mass of greater than 6,000 Da; and/or
(B) a number average molar mass of the first polymer and a number average molar mass of the second polymer are different, further optionally wherein:
   a. a number average molar mass of the second polymer is greater than a number average molar mass of the first polymer; and/or
   b. a number average molar mass of the second polymer is less than a number average molar mass of the first polymer; and/or
(C) the number average molar mass of the first polymer is at least or up to about 1 kilodalton (kDa), at least or up to about 2 kDa, at least or up to about 5 kDa, at least or up to about 7 kDa, at least or up to about 10 kDa, at least or up to about 20 kDa, at least or up to about 50 kDa, at least or up to about 70 kDa, at least or up to about 100 kDa, at least or up to about 200 kDa, at least or up to about 500 kDa, at least or up to about 700 kDa, at least or up to about 1,000 kDa, further optionally wherein:
   a. the number average molar mass of the first polymer is at least about 10 kDa; and/or
   b. the number average molar mass of the first polymer is at most about 100 kDa; and/or
(D) the number average molar mass of the second polymer is at least or up to about 1 kilodalton (kDa), at least or up to about 2 kDa, at least or up to about 5 kDa, at least or up to about 7 kDa, at least or up to about 10 kDa, at least or up to about 20 kDa, at least or up to about 50 kDa, at least or up to about 70 kDa, at least or up to about 100 kDa, at least or up to about 200 kDa, at least or up to about 500 kDa, at least or up to about 700 kDa, at least or up to about 1,000 kDa, further optionally wherein:
   a. the number average molar mass of the second polymer is at least about 10 KDA; and/or
   b. the number average molar mass of the second polymer is at most about 100 kDa; and/or
(E) the first polymer comprises polycaprolactone (PCL), poly lactic acid (PLA), poly L-lactide-glycolic acid (PLGA), and/or polyethylene glycol diacrylate (PEGDA), further optionally wherein the first polymer comprises PCL; and/or
(F) the second polymer comprises polycaprolactone (PCL), poly lactic acid (PLA), poly L-lactide-glycolic acid (PLGA), and/or polyethylene glycol diacrylate (PEGDA),
further optionally wherein the second polymer comprises PCL; and/or
(G) the therapeutic agent comprises an anti-cancer agent, an antimicrobial agent, an antibiotic, a local anesthetic or analgesic, a statin, and/or an anti-inflammatory agent; and/or
(H) the therapeutic agent comprises capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, and/or vinorelbine,
further optionally wherein the therapeutic agent comprises fluorouracil.

### Embodiment 2

A biomaterial comprising a plurality of geometric elements and a therapeutic agent, wherein:
a first geometric element of the plurality of geometric elements comprises a first polymer and the therapeutic agent, wherein an amount of the therapeutic agent in the first geometric element is greater than 30% by weight of the first polymer, and wherein at least a portion of the first geometric element is porous;
a second geometric element of the plurality of geometric elements is formed by a second polymer, wherein at least a portion of the second geometric element is substantially non-porous; and
the at least the portion of the first geometric element is adjacent to the at least the portion of the second geometric element,
optionally wherein:
(A) the amount of the therapeutic agent in the first geometric element is greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or more by weight of the first polymer,
further optionally wherein the amount of the therapeutic agent in the first geometric element is greater than 35% by weight of the first polymer; and/or
(B) the amount of the therapeutic agent in the first geometric element is greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or more by weight of a combination of the first polymer and the second polymer,
further optionally wherein the amount of the therapeutic agent in the first geometric element is greater than 35% by weight of the combination of the first polymer and the second polymer; and/or
(C) the first polymer comprises polycaprolactone (PCL), poly lactic acid (PLA), poly L-lactide-glycolic acid (PLGA), and/or polyethylene glycol diacrylate (PEGDA), further optionally wherein the first polymer comprises PCL; and/or
(D) the second polymer comprises polycaprolactone (PCL), poly lactic acid (PLA), poly L-lactide-glycolic acid (PLGA), and/or polyethylene glycol diacrylate (PEGDA), further optionally wherein the second polymer comprises PCL; and/or
(E) the therapeutic agent comprises an anti-cancer agent, an antimicrobial agent, an antibiotic, a local anesthetic or analgesic, a statin, and/or an anti-inflammatory agent; and/or
(F) the therapeutic agent comprises capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, and/or vinorelbine,
further optionally wherein the therapeutic agent comprises fluorouracil; and/or
(G) the porosity of first geometric element is at least or up to about 1%, at least or up to about 2%, at least or up to about 5%, at least or up to about 7%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, or at least or up to about 99%, as ascertained by scanning electron microscopy, thermoporometry and/or cryoporometry.

### Embodiment 3

A method of treating a condition in a subject in need thereof, the method comprising administering to the subject a biomaterial comprising a plurality of geometric elements and a therapeutic agent, wherein:
a first geometric element of the plurality of geometric elements comprises a first polymer and the therapeutic agent, wherein at least a portion of the first geometric element is porous;
a second geometric element of the plurality of geometric elements comprises a second polymer, wherein at least a portion of the second geometric element is substantially non-porous;
the at least the portion of the first geometric element is adjacent to the at least the portion of the second geometric element; and
the first polymer or the second polymer has a number average molar mass of greater than 6,000 Dalton (Da),
optionally wherein:
(I) the first polymer and the second polymer have a number average molar mass of greater than 6,000 Da; and/or
(J) a number average molar mass of the first polymer and a number average molar mass of the second polymer are different, further optionally wherein:
   a. a number average molar mass of the second polymer is greater than a number average molar mass of the first polymer; and/or
   b. a number average molar mass of the second polymer is less than a number average molar mass of the first polymer; and/or
(K) the number average molar mass of the first polymer is at least or up to about 1 kilodalton (kDa), at least or up to about 2 kDa, at least or up to about 5 kDa, at least or up to about 7 kDa, at least or up to about 10 kDa, at least or up to about 20 kDa, at least or up to about 50 kDa, at least or up to about 70 kDa, at least or up to about 100 kDa, at least or up to about 200 kDa, at least or up to about 500 kDa, at least or up to about 700 kDa, at least or up to about 1,000 kDa,
further optionally wherein:
   a. the number average molar mass of the first polymer is at least about 10 kDa; and/or
   b. the number average molar mass of the first polymer is at most about 100 kDa; and/or
(L) the number average molar mass of the second polymer is at least or up to about 1 kilodalton (kDa), at least or up to about 2 kDa, at least or up to about 5 kDa, at least or up to about 7 kDa, at least or up to about 10 kDa, at least or up to about 20 kDa, at least or up to about 50 kDa, at least or up to about 70 kDa, at least or up to about 100 kDa, at least or up to about 200 kDa, at least or up to about 500 kDa, at least or up to about 700 kDa, at least or up to about 1,000 kDa,
further optionally wherein:
   a. the number average molar mass of the second polymer is at least about 10 KDA; and/or
   b. the number average molar mass of the second polymer is at most about 100 kDa; and/or
(M) the first polymer comprises polycaprolactone (PCL), poly lactic acid (PLA), poly L-lactide-glycolic acid (PLGA), and/or polyethylene glycol diacrylate (PEGDA), further optionally wherein the first polymer comprises PCL; and/or
(N) the second polymer comprises polycaprolactone (PCL), poly lactic acid (PLA), poly L-lactide-glycolic acid (PLGA), and/or polyethylene glycol diacrylate (PEGDA),
further optionally wherein the second polymer comprises PCL; and/or
(O) the therapeutic agent comprises an anti-cancer agent, an antimicrobial agent, an antibiotic, a local anesthetic or analgesic, a statin, and/or an anti-inflammatory agent; and/or
(P) the therapeutic agent comprises capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, and/or vinorelbine,
further optionally wherein the therapeutic agent comprises fluorouracil;
further optionally wherein the condition is cancer, optionally wherein the cancer is a solid tumor, colorectal cancer, rectal cancer, anal cancer, lower gastrointestinal tract cancer, pancreatic cancer, lung cancer, bone cancer, or locally advanced cancer;
further optionally wherein the administering comprises implanting the biomaterial in the subject adjacent to a tumor.

### Embodiment 4

A method of treating a condition in a subject in need thereof, the method comprising administering to the subject a biomaterial comprising a plurality of geometric elements and a therapeutic agent, wherein:
a first geometric element of the plurality of geometric elements comprises a first polymer and the therapeutic agent, wherein an amount of the therapeutic agent in the first geometric element is greater than 30% by weight of the first polymer, and wherein at least a portion of the first geometric element is porous;
a second geometric element of the plurality of geometric elements is formed by a second polymer, wherein at least a portion of the second geometric element is substantially non-porous; and the at least the portion of the first geometric element is adjacent to the at least the portion of the second geometric element:
   optionally wherein:
(H) the amount of the therapeutic agent in the first geometric element is greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or more by weight of the first polymer, further optionally wherein the amount of the therapeutic agent in the first geometric element is greater than 35% by weight of the first polymer; and/or
(I) the amount of the therapeutic agent in the first geometric element is greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or more by weight of a combination of the first polymer and the second polymer, further optionally wherein the amount of the therapeutic agent in the first geometric element is greater than 35% by weight of the combination of the first polymer and the second polymer; and/or
(J) the first polymer comprises polycaprolactone (PCL), poly lactic acid (PLA), poly L-lactide-glycolic acid (PLGA), and/or polyethylene glycol diacrylate (PEGDA),
further optionally wherein the first polymer comprises PCL; and/or
(K) the second polymer comprises polycaprolactone (PCL), poly lactic acid (PLA), poly L-lactide-glycolic acid (PLGA), and/or polyethylene glycol diacrylate (PEGDA),
further optionally wherein the second polymer comprises PCL; and/or
(L) the therapeutic agent comprises an anti-cancer agent, an antimicrobial agent, an antibiotic, a local anesthetic or analgesic, a statin, and/or an anti-inflammatory agent; and/or
(M) the therapeutic agent comprises capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, and/or vinorelbine,
further optionally wherein the therapeutic agent comprises fluorouracil; and/or
(N) the porosity of first geometric element is at least or up to about 1%, at least or up to about 2%, at least or up to about 5%, at least or up to about 7%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, or at least or up to about 99%, as ascertained by scanning electron microscopy, thermoporometry and/or cryoporometry;
further optionally wherein the therapeutic agent comprises fluorouracil;
further optionally wherein the condition is cancer, optionally wherein the cancer is a solid tumor, colorectal cancer, rectal cancer, anal cancer, lower gastrointestinal tract cancer, pancreatic cancer, lung cancer, bone cancer, or locally advanced cancer;
further optionally wherein the administering comprises implanting the biomaterial in the subject adjacent to a tumor.

1. A biomaterial comprising a plurality of geometric elements and a therapeutic agent, wherein:
(a) a first geometric element of the plurality of geometric elements comprises a first polymer and the therapeutic agent, wherein at least a portion of the first geometric element is porous;
(b) a second geometric element of the plurality of geometric elements comprises a second polymer, wherein at least a portion of the second geometric element is substantially non-porous;
(c) the at least the portion of the first geometric element is adjacent to the at least the portion of the second geometric element; and
(d) the first polymer or the second polymer has a number average molar mass of greater than 6,000 Dalton (Da).

2. The biomaterial of claim 1, wherein the first polymer and the second polymer have a number average molar mass of greater than 6,000 Da.

3. The biomaterial of claim 1, wherein the number average molar mass of the first polymer and the number average molar mass of the second polymer are different.

4-6. (canceled)

7. The biomaterial of claim 1, wherein the number average molar mass of the first polymer is at least about 10 kDa.

8. The biomaterial of claim 1, wherein the number average molar mass of the first polymer is at most about 100 kDa.

9. (canceled)

10. The biomaterial of claim 1, wherein the number average molar mass of the second polymer is at least about 10 kDa.

11. The biomaterial of claim 1, wherein the number average molar mass of the second polymer is at most about 100 kDa.

12. The biomaterial of claim 1, wherein the first polymer comprises polycaprolactone (PCL), poly lactic acid (PLA), poly L-lactide-glycolic acid (PLGA), or polyethylene glycol diacrylate (PEGDA).

13. The biomaterial of claim 1, wherein the first polymer comprises PCL.

14. The biomaterial of claim 1, wherein the second polymer comprises polycaprolactone (PCL), poly lactic acid (PLA), poly L-lactide-glycolic acid (PLGA), or polyethylene glycol diacrylate (PEGDA).

15. The biomaterial of claim 1, wherein the second polymer comprises PCL.

16. The biomaterial of claim 1, wherein the therapeutic agent comprises an anti-cancer agent.

17. The biomaterial of claim 1, wherein the therapeutic agent comprises an antimicrobial agent, an antibiotic, a local anesthetic or analgesic, a statin, and/or an anti-inflammatory agent.

18. The biomaterial of claim 1, wherein the therapeutic agent comprises capecitabine, cisplatin, carboplatin, cyclophosphamide, docetaxel, doxorubicin, etoposide, fluorouracil, floxuridine, gemcitabine, ifosfamide, irinotecan, methotrexate, oxaliplatin, paclitaxel, pemetrexed, raltitrexed, regorafenib, vincristine, or vinorelbine.

19. The biomaterial of claim 1, wherein the therapeutic agent comprises fluorouracil.

20. The biomaterial of claim 1, wherein an amount of the therapeutic agent in the first geometric element is greater than 30% by weight of the first polymer.

21. (canceled)

22. The biomaterial of claim 20, wherein the amount of the therapeutic agent in the first geometric element is greater than 35% by weight of the first polymer.

23. The biomaterial of claim 20, wherein the amount of the therapeutic agent in the first geometric element is greater than 5% by weight of a combination of the first polymer and the second polymer.

24. (canceled)

25. The biomaterial of claim 20, wherein the amount of the therapeutic agent in the first geometric element is greater than 35% by weight of the combination of the first polymer and the second polymer.

26-33. (canceled)

34. The biomaterial of claim 20, wherein the porosity of first geometric element is at least about 10% as ascertained by scanning electron microscopy, thermoporometry and/or cryoporometry.

35-90. (canceled)
